(19) United States
(12) Patent Application Publication
(10) Pub. No.: US 2009/0291048 A1
(43) Pub. Date: Nov. 26, 2009

(54) LEUKOCYTE-BINDING POLYPEPTIDES AND USES THEREOF

(75) Inventors: Karlheinz Peter, Victoria (DE); Steffen Eisenhardt, Victoria (AU); Meike Schwarz, Freiburg (DE)

Correspondence Address: CLARK & ELBING LLP, 101 FEDERAL STREET, BOSTON, MA, 02110, US

(73) Assignee: Baker IDI Heart and Diabetes Institute Holdings Limited, Melbourne, Victoria (AU)

(21) Appl. No.: 12/091,708

(22) PCT Filed: Oct. 25, 2006
(86) PCT No.: PCT/AU2006/001586

§ 371 Date: Feb. 23, 2009

(30) Foreign Application Priority Data
Oct. 25, 2005 (AU) 2005905904

(51) Int. Cl. A61K 51/00; C07K 7/00; C07K 16/28; A61K 39/395; G01N 33/53; A61K 49/00; C40B 30/04

(52) U.S. Cl. 424/1.49; 530/328; 530/387.3; 424/133.1; 435/7.21; 424/9.1; 424/9.3; 424/9.6; 424/9.5; 506/9

(57) ABSTRACT

The present invention provides molecules capable of specifically binding the activated form of the beta-integrin Mac-1. The molecules may be provided in the form of peptides, polypeptides and single chained antibodies. The molecules may be used therapeutically for the treatment of disease mediated by Mac-1 (such as inflammation), or used diagnostically to locate sites of Mac-1 activity in the body.

The present invention relates to the field of medical immunology. More specifically, the invention relates to the modulation of pathways mediated by leukocytes.

### BACKGROUND TO THE INVENTION

Inflammation is a complex process of the immune system, having a cellular component and an exudative component. The exudative component involves the movement of fluid, usually containing proteins such as fibrin and immunoglobulins. Blood vessels dilate upstream of an infection (causing redness and heat) and constrict downstream, while capillary permeability to the affected tissue is increased resulting in a net loss of fluid to the tissue, thereby giving rise to edema.

The cellular component of inflammation is more complex, requiring the movement of leukocytes out of the capillaries and into the surrounding tissue beds, where they act as phagocytes inactivating bacteria and collecting cellular debris. Where inflammation of the affected site persists, cytokines such as IL-1 and TNF are released to activate many cell types leading to the upregulation of receptors such as VCAM-1, ICAM-1, E-selectin, and L-selectin. Receptor upregulation increases extravasation of neutrophils, monocytes, activated T-helper and T-cytotoxic, and memory T and B cells to the infected site.

While inflammation is clearly important in the normal functioning of the immune system, it can also lead to significant morbidity in the subject. For example, connective tissue scarring can be the result. Some 24 hours after inflammation first occurred the healing response will commence, this response involves the formation of connective tissue to bridge the gap caused by injury, and the process of angiogenesis which is the formation of new blood vessels, to provide nutrients to the newly formed tissue. Often healing cannot occur completely and a scar will form; for example after laceration to the skin, a connective tissue scar results which does not contain any specialized structures such as hair or sweat glands. Connective tissue scarring may also lead to the formation of adhesions between various tissues and organs.

Frequently, the inflammatory response does not self-limit and ongoing or chronic inflammation results. This is marked by inflammation lasting many days, months or even years. It is characterized by a dominating presence of macrophages in the injured tissue, which extravasate via the same methods discussed above (ICAM-1 VCAM-1). These cells are powerful defensive agents of the body, but the toxins they release (including reactive oxygen species) are injurious to bodily tissues as well as pathogens. Thus, chronic inflammation is almost always accompanied by tissue destruction.

Of particular concern, inflammation can lead to a number of diseases such as rheumatoid arthritis, multiple sclerosis, asthma, Crohn's disease, and the like. Inflammation is also involved in other processes such as ischaemia and infarction.

Given the complexity and clinical importance of inflammatory pathways, a significant amount research has been devoted to elucidating the molecular mechanisms involved, and also to identifying molecules capable of modulating inflammation. While the prior art has provided a range of polypeptides and antibodies capable of modulating inflammation (e.g. Enbrel®, a TNF-alpha binding monoclonal antibody), alternatives are still required to target different mediators of inflammation. It is an aspect of the present invention to overcome or alleviate a problem of the prior art by providing a modulator of inflammation useful in therapy or prophylaxis of inflammatory conditions.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

### SUMMARY OF THE INVENTION

Applicants have identified molecules that are capable of binding to an activated form of the Mac-1 receptor molecule. Mac-1 is the main integrin receptor molecule expressed on the surface of phagocytic leukocytes such as neutrophils and monocytes. Accordingly, a first aspect the present invention provides a non-natural molecule capable of binding to activated Mac-1. In a preferred form of the invention the molecule is a peptide, polypeptide or derivative thereof including the amino acid sequence motif DX₁X₂X₃X₄X₅X₆ X₇X₈X₉Y, wherein X₁ is S or no amino acid; X₂ is independently T, L or F; X₃ is independently L or W; X₄ is independently A or G; X₅ is independently P, F or no amino acid; X₆ is Q or no amino acid; X₇ is independently I, L or S; X₈ is independently F or Y; and X₉ is independently E or D.

Significantly, Applicants have also demonstrated for the first time a non-natural molecule that specifically binds preferentially the activated form of the receptor, with little or no affinity for the non-activated form. Furthermore, in one form of the invention the molecule is substantially incapable of interfering with the binding of C3bi to Mac-1.

Preferably, the molecule, peptide, polypeptide or derivative is capable of interfering with the binding of a ligand to Mac-1 selected from the group consisting of intracellular adhesion molecule-1 (ICAM-1), fibrinogen (Fg), Factor Xa, heparin, GPIb-alpha, JAM-3, lipoprotein (a), and denatured proteins.

The peptide, polypeptide or derivative of the invention may take a number of forms, however the dipeptide WG has been shown by alanine scanning to play a role in the ability of the polypeptide or derivative to bind one epitope of the activated form of Mac-1 (see Example 3). The same Example shows that it is possible to substitute residues surrounding the WG residues without materially affecting the ability of the polypeptide or derivative to bind Mac-1.

In a highly preferred form of the invention the peptide, polypeptide or derivative includes the amino acid sequence DLWGFQLFDY, DFWGSYDY, or DSTLAPIFEY.

The skilled person will understand that once provided with the inventive sequences described supra, it will be possible to modify the residues and sequences to provide a peptide, polypeptide or derivative without totally destroying the ability to bind to activated Mac-1.

In a further preferred form of the invention the polypeptide or derivative is in the form of a single-chain antibody molecule. In a more highly preferred form of the invention, the single chain antibody includes one or more of the following regions HCDR1, HCDR2, HCDR3, LINKER, LCDR1, LCDR2, LCDR3. In one embodiment, the HCDR1 is MSGFIFRDYDMD or MSGFSNYGIH or equivalent sequence, the HCDR2 is independently RSTKRTSSYTIQDAA or VALISYDNGNKKFYA or equivalent sequence, the HCDR3 region is independently DLWGFQLFDY, DFWGSYDY or DSTLAPIFEY or equivalent sequence, the LINKER is independently KLEEGEFSEARV or equivalent sequence, the LCDR1 is independently GGNNIGSKSVH or GGNNIGSTTVH or equivalent sequence, the LCDR2 is independently YDSVRPS or DDNERPS or equivalent sequence, the LCDR3 is independently QVWDSNTDHYV or QVWDSGSDHW or equivalent sequence.

Single chain antibodies used as therapeutics provide high tissue penetration, fast clearance (often useful for high tumor to healthy tissue ratio and certain acute-care applications), renal clearance depending on their engineered size (avoiding potential dose limiting effects that otherwise might come from hepatotoxicity), and no intrinsic effector function thereby limiting potential immunogenicity issues.

Given the biological activity of the polypeptides and derivatives described herein, the present invention further provides a composition including a molecule, peptide, polypeptide or derivative as described herein in and a pharmaceutically acceptable carrier.

In another embodiment of the present invention provides a method of treating a condition associated with inflammation in a patient in need of such therapy comprising administering to the patient an effective amount of a pharmaceutical composition comprising at least one molecule, peptide, polypeptide or derivative thereof as described herein, wherein the molecule, peptide, polypeptide or derivative thereof is capable of specific binding with the Mac-1 receptor. Inflammation mediated diseases include, but are not limited to Crohn's disease, collitis ulcerosa, multiple sclerosis, sarcoidosis, psoriasis, atherosclerosis and its clinical sequelae, scleroderma, intestinal adhesions, hypertrophic scars, rheumatoid arthritis, septicemia, autoimmune disease, acute coronary syndrome, HIV infection, reperfusion injuries, ischemia, neointimal thickening, infiltration of polymorpholeucocytes, autoimmune disease, and neovascularisation-mediated diseases.

The present invention may also be used in a diagnostic setting to detect the presence, absence or level of inflammation. Accordingly, the invention further provides a method for detecting the presence, absence or level of an activated Mac-1 in a subject or a test article, the method including exposing the subject, or a biological sample of the subject or the test article, to a molecule, peptide, polypeptide or derivative thereof as described herein, and detecting binding of the molecule, peptide, polypeptide or derivative thereof to activated Mac-1. Typically, the molecule, peptide or polypeptide is tagged such that it is detectable by an imaging technique such as MRI or gamma scintigraphy.

In another aspect, the present invention provides a method of diagnosis or prognosis of a Mac-1 mediated condition, the method including a method for detecting the presence, absence or level of an activated Mac-1 in a subject as described herein. In one form of the method, the Mac-1 related condition is sepsis.

In a further aspect the present invention provides a method for identifying a molecule capable of binding to activated Mac-1, the method including the steps of providing a library of candidate molecules, providing a first cell type exhibiting either activated Mac-1 or non-activated Mac-1, providing a second cell type exhibiting either activated Mac-1 or non-activated Mac-1, exposing the library of candidate molecules to the first cell type exhibiting non-activated Mac-1 and removing bound molecules to leave a first pool of molecules, exposing the first pool of molecules to the first cell type exhibiting activated Mac-1 and removing unbound molecules to leave a second pool of molecules, exposing the second pool of molecules to the second cell type exhibiting non-activated Mac-1 and removing unbound molecules to leave a third pool of molecules, exposing the third pool of molecules to the second cell type exhibiting activated Mac-1 and removing the unbound molecules to leave a fourth pool of molecules.

### BRIEF DESCRIPTION OF THE FIGURES

FIG. 1 shows a schematic of a differential panning method used to isolate phage capable of binding to activated Mac-1 in preference to non-activated Mac-1. This novel strategy was developed to deplete phages that bind non-specific (depicted in gray) and/or non-activation-specific (depicted in blue) to Mac-1 or other surface molecules and to specifically select phages that bind to epitopes on Mac-1 expressed solely on the activated receptor (depicted in red). The first round (upper row) was performed on human monocytes. Initially, a depletion step was performed where all phages that bound to non-activated Mac-1 or to the monocytes cell surface were separated by centrifugation and discarded. The supernatant was used for the next selection step in which the non-binding phages in the supernatant were discarded and the binding phages were rescued and eluted by lowering the pH. The obtained phages were then amplified in E. coli and used for the next round. In the following two rounds the cell background was changed to Mac-1-expressing CHO cells to avoid enrichment of phages binding to activation-specific monocyte epitopes others than those on the Mac-1 integrin.

FIG. 2a shows the results of four rounds of panning using the basic scheme outlined in FIG. 1. After each round of panning the rescued phages were used for infection of log-phase XL-1 blue E. coli bacteria, which were plated on 14 cm agar plates containing 50 mM glucose, 100 μg/ml ampicillin and 20 μg/ml tetracycline and grown over night. The number of colonies, which are representing the number of clones were counted. The increase of clones after panning round 4 represents the amplification of a few very strongly binding clones. The x-axis shows two groups of bars: those on the left reflect phage expressing peptides from a natural library, those on the right reflect phage expressing peptides from a synthetic library. Within each group of bars, the individual bars represent the number of eluted clones after 1, 2, 3, or 4 rounds of panning. The number of eluted clones is represented on the y-axis.

FIG. 2b shows fingerprinting of natural clones by BSTN-1 digest. Phagemid-DNA of 10 randomly picked natural clones was purified and digested with the BstNI restriction enzyme and separated in electrophoresis and stained with ethidium bromide showing the same restriction pattern for all 10 clones, indicating, that only one clone was amplified over the course of panning. The two outermost lanes are molecular weight markers. Lanes 2 to 11 represent different natural clones.

FIG. 2c shows fingerprinting of natural clones by BstN I and Rsa I digest: The diversity of the natural clones was evaluated by digestion with the restriction enzymes BstN I and Rsa I. Phagemid DNA of 20 randomly picked natural clones of panning round 2, 3 and 4 was purified and digested with the restriction enzymes and separated in agarose-gel-electrophoresis and stained with ethidiumbromide. The DNA markers Lambda DNA/Hind III (lane 1) and PhiX174DNA/Hae III (lane 2) are used as comparison. The restriction pattern of scFv clones differs widely after panning round 2 and 3. In contrast, after panning round 4 all investigated clones demonstrate an identical restriction pattern. These results demonstrate the power of positive clone amplification using the developed depletion/selection system over the course of consequent panning rounds. In the case of the synthetic library, where restriction pattern analysis does not work because of sequence identity outside the HCDR3 region, 10 randomly picked clones were sequenced, revealing two distinct clones, each represented 5 times.

FIG. 2d shows MAN-1 production and purification. (left) Silver staining of SDS-PAGE. (right) Western blot probed with an anti-HIS-tag HRP-coupled antibody. Phagemid DNA was cloned into the expression vector pHOG-21 using the restriction enzymes Nco I and Not I and transformed into TG-1 E. coli. These bacteria were grown at 37° C. to an optical density of 0.8 in LB-medium containing glucose (50 mM) and 100 μg/ml ampicillin. Then, bacteria were transferred to LB-medium containing 0.4M sucrose, 100 μg/ml ampicillin and 0.25 mM IPTG and incubated for 16 h at 200 rpm, 23° C. For the isolation of the periplasma, bacteria were centrifuged at 3000 g for 10 min and resuspended in 5 ml per mg of pellet 1× BugBuster® (Novagen) solution. After 30 min incubation at room temperature and centrifugation for 30 min at 10,000 g at 4° C. the supernatant containing the periplasmic proteins was run over a Ni-NTA-agarose-column (Quiagen). The column was washed twice with washing buffer containing 50 mM NaH₂PO₄, 300 mM NaCl and 20 mM imidazole, pH 8.0 and then eluted with 500 μl elution buffer per liter bacterial culture, containing 50 mM NaH₂PO₄, 300 mM NaCl, 250 mM imidazole, pH 8.0. Subsequently, the purified protein was dialyzed against PBS in Slide-A-Lyzer® Dialysis cassettes with a molecular mass cut off at 10 000 Dalton.

Production and purification were monitored by SDS-PAGE with silver staining (left) and Western blotting (right). Gel and blot show:

lane I: bacterial culture
lane II: supernatant of the first centrifugation step
lane III: lysate after BugBuster® treatment of the bacterial pellet
lane IV: flow through of the Ni-NTA-agarose-column
lane V: first wash out of the Ni-NTA-agarose-column
lane VI: second wash out of the Ni-NTA-agarose-column
lane VII: empty
lane VIII: eluate of the Ni-NTA-agarose-column

FIG. 3a shows the amino acid sequences of the clones MAN-1, MAS-1 and MAS-2 (top to bottom). “X” denotes an amino acid residue that cannot be definitively identified

FIG. 3b shows a sequence alignment of the HCDR3 regions of MAN-1, MAS-1 and MAS-2.

FIG. 4 shows the results of an alanine scan of the HCDR3 region of MAN-1. From top to bottom, the relevant sequences are CARDWGSTDY (wild type), CARAFWGSYDY (D to A substitution), CARDAWGSYDY (F to A substitution), CARDFAGSYDY (W to A substitution), CARDFWASYDY (G to A substitution), CARDFWGAYDY (S to A substitution). The x-axis shows binding to monocytes by mean fluorescence. The binding properties of the MAN-1 HCDR3 mutants were determined by flow cytometry using an Alexa Fluor 488 conjugated anti-His-tag antibody.

FIG. 5a shows binding of distinct antibodies to Mac-1-expressing CHO cells. The graphs in the left hand column (Panels A, C and E) show wild type Mac-1, while those on the right (Panels B, D and F) show “activated” (GFFKR-deleted) Mac-1. The first row (Panels A and B) shows IgG anti-CD18, the second row (Panels C and D) shows IgG anti-CD11b, and the third row (Panels E and F) shows scFv MAN-1. Binding of the scFv MAN-1 were detected by an Alexa Fluor 488 conjugated anti-His tag antibody. The CD11b and CD 18 antibodies were directly FITC-labeled.

FIG. 5b shows Binding of MAN-1 to monocytes in whole blood by flow cytometry. The trace in the lightest line shows control (an unspecific scFv), the medium line shows resting monocytes, with the dark line showing PMA-stimulated monocytes. Binding of the scFvs is detected by an Alexa Fluor 488-conjugated anti-His(6)-tag antibody. The x-axis is FL1-Height, and the y-axis shows counts.

FIG. 5c shows a titration curve of MAN-1 on resting monocytes (no addition of PMA; light line) compared with PMA-stimulated monocytes (100 ng/ml PMA; dark line). X-axis: MAN-1 concentration (ug/ml); y-axis: mean fluorescence.

FIG. 6 shows binding of scFv MAN-1 to an I-domain peptide (KFGDPLGY EDVIPEADR) as evaluated by ELISA. The left bar shows MAN-1, the right bar shows control. Binding was measured by an anti-His(6)-tag antibody and an anti-mouse mAb HRP-conjugate. A scFv that does not bind to Mac-1 was used as a negative control. After reaction with TMB-Substrate absorption was read in an ELISA plate reader at 450 nm. Mean and standard deviation of triplicate experiments are given. Absorbance at 450 nm is shown on the y-axis.

FIG. 7 shows inhibition of ligand binding by scFv MAN-1 in static adhesion.

FIG. 7a shows binding to fibrinogen, and FIG. 7b shows binding to heparin. For each panel, the pairs of bars running left to right correspond to no ligand, antiCD11b (10 ug/ml), MAN-1 (10 ug/ml), unspecific antibody (10 ug/ml). Within each pair of bars, the light bar corresponds to CHO cells, and the left bar corresponds to GFFKR-deleted Mac-1-expressing CHO cells. After pre-incubation with either blocking anti-CD1b mAb as positive control or the activation-specific scFv MAN-1, adhesion of CHO cells transfected with the GFFKR-deleted and thereby activated Mac-1 receptor to immobilized fibrinogen and heparin was evaluated. Non-transfected CHO cells served as negative control. Cell adhesion was measured using a calorimetric with readings at 562 nm (y-axis). Mean and standard deviation is given for triplicate experiments.

FIG. 7c and FIG. 7d. shows static adhesion of Mac-1-expressing CHO cells to ICAM-1-expressing CHO cells is inhibited by MAN-1, whereas adhesion to immobilized C3bi is not inhibited. Cells expressing the GFFKR-deleted, activated Mac-1 adhere stronger to immobilized C3bi than non-activated Mac-1 cells or a CHO cell control. Binding can be inhibited by an activation-unspecific anti-Mac-1 antibody, but not by scFv MAN-1. Adherent cells were quantified with a phosphatase-substrate assay and absorbance was read at 405 nm. Mean and standard deviation is given for triplicate experiments. Adhesion of Mac-1-expressing CHO cells to immobilized ICAM-1-expressing CHO cells were counted based on their clearly distinguishable round shape on a flat monolayer of ICAM-1-expressing cells. 6 visual fields were counted. Experiments were performed in triplicates. All static adhesion assays were performed at least 5 times. Representative results are shown.

FIG. 8a shows activation-specific inhibition of recombinant Mac-1 under conditions of blood flow by scFv MAN-1. Panels on left reflect venous flow (0.5 dynes/cm²), while those on the left reflect arterial flow (15 dynes/cm²). By columns 1 to 4, columns 1 and 3 show native Mac-1 while columns 2 and 4 show GFFKR-deleted Mac-1. By row, row 1 is control, row 2 is MAN-1, row 3 is CD11b. Row 4 are graphs showing quantitation of the panels directly above. The lightest bars show control, the medium bars show MAN-1, the darkest bars show CD11b antibody. The results show ScFv MAN-1 effectively inhibits adhesion of CHO cells expressing activated Mac-1 but not native Mac-1 on immobilized fibrinogen under flow conditions. A Mac-1-blocking mAb was used as a negative control.

FIG. 8b shows activation specific inhibition on monocytes under conditions of blood flow by scFv MAN-1. Panels on left reflect venous flow (0.5 dynes/cm²), while those on the left reflect arterial flow (15 dynes/cm²). By columns 1 to 4, columns 1 and 3 show non-activated cells, while columns 2 and 4 show PMA-stimulated cells. By row, row 1 is control, row 2 is MAN-1. Row 3 are graphs showing quantitation of the panels directly above. For each graph, the first group of three bars relate to unstimulated cells, while the second group of three bars are PMA stimulated cells. The lightest bars show control, the medium bars show MAN-1, the darkest bars show CD11b antibody. This figure shows that ScFv MAN-1 effectively inhibits adhesion of PMA-activated monocytes but not non-activated monocytes on immobilized fibrinogen under flow conditions.

FIG. 8c shows inhibition of adhesion of Mac-1-expressing CHO cells on immobilized fibrinogen by circular MAS-1 and MAS-2 derived peptides under flow conditions. For all flow experiments, mean and standard deviation of adhering cells based on the counting of 5 visual fields are given. Representative examples of at least 6 experiments are demonstrated.

FIG. 9. shows PCR primers used for the site directed mutations of selected amino acids in the HCDR3 domain of MAN-1, MAS-1 and MAS-2.

FIG. 10a shows MAN-1 does not exhibit cross-reactivity with the β2-integrins LFA-1 (α_{L}β₂, CD11a/CD18), p150/95 (α_{X}β₂, CD11c/CD18), α_{D}β₂ (CD11d/CD18). MAN-1 cross-reactivity with β2-integrins others than Mac-1 was investigated in flow cytometry. Leukocytes in whole blood were activated and MAN-1 binding was evaluated as described in the Examples. Blocking antibody clones TS1 (CD11a, ATCC), 2LPM19c (CD11b, DAKO), BU15 (CD11c, Serotec), and 2401 (CD11d, kindly provided by ICOS, Bothell, Wash.) were added in various concentrations and incubated for 15 min. Subsequently MAN-1 was added at a concentration of 5 μg/ml for 10 min and binding was detected by an Alexa Fluor 488-conjugated anti-His-tag secondary antibody. In parallel 132-integrin expression was assessed with the same antibodies and a secondary FITC-coupled goat-anti-mouse antibody. The FITC-coupled goat-anti-mouse antibody alone served as a control. Measurements were performed in triplicates. A typical result out of three experiments is shown. The blocking anti-CD11b antibody reduces MAN-1 binding to the background level at a concentration of 50 μg/ml. In contrast, at the same concentrations the other blocking antibodies did not inhibit MAN-1 binding. These findings imply a selective binding of MAN-1 to Mac-1 without cross-reactivity to other β2-integrins.

FIG. 10b shows MAN-1 does not exhibit cross-reactivity to GPIIb/IIIa (α_{IIb}β₃, CD41/CD61). Cross-reactivity with another fibrinogen binding integrin was assessed in flow cytometry using CHO cells expressing either a GFFKR-deleted and thereby activated GPIIb/IIIa or the native and thereby non-activated platelet integrin GPIIb/IIIa (details of cell generation have been described previously).^{30,31} The expression of the activated conformation was determined by the binding of the activation-specific antibody Pac-1. MAN-1 binding was measured by an Alexa Fluor 488-conjugated anti-His-tag secondary antibody. MAN-1 binds neither to non-activated, nor activated GPIIb/IIIa. Thus, no cross-reactivity to GPIIb/IIIa was seen. A typical result out of three experiments is shown.

FIG. 10c shows scFv MAN-1 immunoprecipitates the Mac-1 complex CD11b/CD18 as detected by silver-staining. Lysed monocytes were incubated with either 10 μg/ml MAN-1 with anti-His(6)tag antibody (Novagen) or 10 μg/ml anti-CD11b antibody clone 2LPM19c (Dako). Subsequently, Protein G sepharose beads (Zymed) were used to precipitate bound proteins. Samples were run on SDS-PAGE and the gel was stained by silver-stain (Bio-Rad). Size was assessed by a Kaleidoscope marker (Bio-Rad). Both antibodies show similar bands at ˜170 kDA for the CD11b subunit and ˜95 kDA for the CD18 subunit of the Mac-1 receptor. There are no other bands visible in the precipitation. In particular, bands for CD11c (˜150 kDA) or CD11d (˜125 kDA) were not visible in the silver-staining.

FIG. 10d shows scFv MAN-1 immunoprecipitates CD11b from monocyte lysates, but not CD11a, CD11c or CD11d. Lysed monocytes were incubated with 10 μg/ml MAN-1 and anti-His(6) antibody (Novagen). Subsequently Protein G sepharose beads (Zymed) were used to precipitate bound proteins. Samples were run on SDS-PAGE, western blotted on a nitrocellulose membrane (Millipore) and stained for either CD11a (clone 25.3.1, Immunotech), CD11b (Santa Cruz), CD11c (clone BU15, Serotec) or CD11d (clone 169A, ICOS) and detected by secondary goat-anti-mouse HRP (Pierce) antibody for CD11a, CD11c and CD11d or donkey-anti-goat HRP (Santa Cruz) for CD11b. A chemiluminnescence substrate (Pierce) was used to make the bands visible. Results were documented on a Chemidoc Imager (Bio Rad) and analysed by Quantity One software. MAN-1 is able to precipitate CD11b. Faint bands for CD11a, CD11c and CD11c are only visible in the monocyte lysate, but not in the MAN-1 immunoprecipitation. A typical result out of 3 immunoprecipitations is shown.

FIG. 11 shows binding of C3bi to activated Mac-1 can be inhibited by a blocking anti-CD11b antibody, but not by MAN-1. 10 μg/ml C3bi were incubated with activated monocytes and binding was detected by a biotinylated C3bi antibody and avidin-PE in flow cytometry. Blocking antibody clone 2LPM19c inhibits C3bi binding, whereas MAN-1 and a control antibody (against CD7) did not inhibit C3bi binding. The experiment was performed in triplicates. One representative result out of 3 experiments is shown.

FIG. 12 shows MAN-1 binds to immobilized Mac-1 I-domain peptide. A scrambled I-domain peptide, which contained the same amino acids as the I-domain peptide in a randomized order served as control. ScFv binding was detected with an anti-His-tag antibody and a secondary goat anti-mouse HRP antibody. Mean and standard deviation of triplicate experiments are given. A representative example of four experiments is given.

FIG. 13 shows MAN-1 binds as diagnostic marker for basal monocyte activation in patients with sepsis. MAN-1 binding to monocytes of 18 patients with severe sepsis compared to sex and age-matched patients without any sign of inflammation as analyzed by whole blood flow cytometry. MAN-1 binding without activation (basal activation) and after PMA stimulation are shown as percentage of monocytes positive for MAN-1 as detected by an Alexa Fluor 488-labeled anti-His-tag antibody. Significance level p and ns for non-significant are given.

FIG. 14 shows MAN-1 inhibits binding of activated monocytes to immobilized human endothelial cells under shear flow conditions. Monocytes were passed over human microvascular endothelial cells (HMEC) under venous flow conditions and adherent cells were counted.

### DETAILED DESCRIPTION OF THE INVENTION

Applicants have identified non-natural molecules that are capable of binding to the activated form of the Mac-1 receptor molecule. Mac-1 is the main integrin receptor molecule expressed on the surface of phagocytic leukocytes such as neutrophils and monocytes. Applicants have also shown that the molecules are substantially incapable of binding to non-activated Mac-1. This property is significant since the presence of activated Mac-1 is important in important pathways, such as inflammation. Thus, the ability to detect or block activated Mac-1 is contemplated to have significant utility in the diagnosis and treatment of a Mac-1 mediated condition such as inflammation.

Preferably the molecule is a peptide or polypeptide including the amino acid sequence motif DX₁X₂X₃X₄X₅X₆X₇X₈X₉Y, wherein X₁ is S or no amino acid; X₂ is independently T, L or F; X₃ is independently L or W; X₄ is independently A or G; X₅ is independently P, F or no amino acid; X₆ is Q or no amino acid; X₇ is independently I, L or S; X₈ is independently F or Y; and X₉ is independently E or D.

Applicants have used a novel method of screening a phage library (as discussed infra) to identify polypeptides having the motif above capable of binding preferentially to activated Mac-1. As mentioned, Mac-1 (also known as CD11b/CD18, alphaMbeta2, or CR3) is involved in various pathophysiological processes like inflammation, atherosclerosis and ischemia and is thus takes an important role in multiple diseases such as myocardial infarction, septicaemia, and rheumatoid arthritis. Generally, Mac-1 is only able to participate in inflammatory pathways when in the activated form. Hence, a diagnostic reagent for the activated conformational state of Mac-1 will be a useful tool for the further investigation of the mechanisms involved in Mac-1 activation, as well as for the evaluation of Mac-1 function in various clinical conditions. Furthermore, an activation-specific blocker of Mac-1 function is a promising therapeutic agent, allowing a highly specific blockade of monocytes and neutrophils only when those are activated. This may allow for the specific inhibition of inflammatory processes without affecting the overall function of leukocytes.

As used herein, the term “activated” when used in relation to Mac-1 is intended to include any form of the molecule that is capable of reacting with a ligand involved in an inflammatory process. Mac-1 is a chemoattractant activation-dependent molecule that undergoes a conformational change upon stimulation. Mac-1 has been classified as an I-domain integrin, as it contains a so-called I-domain as the typical ligand binding site. The I-domain is not accessible until the receptor performs a conformational change. Even though the binding sites of most ligands have been described, the detailed conformational changes involved in receptor activation have not yet been completely elucidated.

Until stimulation occurs, Mac-1 remains in a resting, non-adhesive state. However, once activated the molecule binds to many ligands in vivo often leading to a detrimental inflammatory process. Preferably, the peptide, polypeptide or derivative is capable of interfering with the binding of a ligand to Mac-1 selected from the group consisting of intracellular adhesion molecule-1 (ICAM-1), fibrinogen (Fg), Factor Xa, heparin, GPIb-alpha, JAM-3, lipoprotein (a), and denatured proteins.

As used herein, “binding” refers to the ability of a given molecule to interact with a receptor such that the interaction between the molecule and the Mac-1 receptor is relatively specific. Therefore, the term “binding” does not encompass non-specific binding, such as non-specific adsorption to a surface. Non-specific binding can be readily identified by including the appropriate controls in a binding assay. Methods for determining the binding affinity are described in the Examples below.

In one form of the invention the binding to activated Mac-1 has a sufficient level of specificity such that the molecule is substantially incapable of binding to a non-Mac-1 integrin molecule (whether activated or not). Applicant proposes that the specific blockade of activated Mac-1 provides advantages that translate into clinical benefits compared to the unselective blockade of Mac-1. One of the Mac-1 natural ligands that demonstrate a differential effect of activation-specific blockade is fibrinogen. In contrast to soluble fibrinogen, immobilized fibrinogen can mediate cell adhesion by binding to non-activated Mac-1. As demonstrated herein, blockade by an activation-specific Mac-1 scFv leaves this Mac-1 function intact, whereas antibodies blocking the activated and the non-activated Mac-1 inhibit Mac-1-mediated cell adhesion on immobilized fibrinogen under static and under flow conditions. The interaction between Mac-1 and the ligand fibrinogen is proposed to be an important mediator of inflammation. Mice carrying a mutated P2C allele of fibrinogen, the major recognition site for the α_{M} I-domain, showed a severely compromised host defence. Single chain variable fragment (ScFv) MAN-1 (MAN-1: Mac-1 activation-specific scFv obtained from the natural library) is directed to the same site on Mac-1 as is fibrinogen, but in contrast to the fibrinogen mutant mice, cell adhesion to immobilized fibrinogen is still possible and the compromise of the immune system may be less. Indeed, experiments with Mac-1 knock-out mice established a pivotal role of this integrin in host defense.

Notably, the phagocytosis of bacteria (e.g. Borrelia burgdorferi) can be mediated by Mac-1 in a non-activated state either via C3bi or by direct interaction between Borrelia burgdorferi outer surface protein and Mac-1. Furthermore, data presented herein show that MAN-1 doesn't interfere with C3bi binding to the activated Mac-1 receptor. These results are consistent with the observation, that the C3bi binding region within the I-domain is not identical with the region for ICAM-1 and fibrinogen. Blocking antibodies that reduce binding to fibrinogen and ICAM-1 inhibit C3bi binding only slightly. Also mutational analysis suggests a different binding site in Mac-1 for fibrinogen and C3bi.

As a component of the compliment system C3b is essentially the last step of the cascade involving C3, and is the unactive conformation of C3b. It “marks” bacterial cells and debris to be phagocytosed by monocytes/macrophages. This happens through the interaction with Mac-1. The fact that the molecules described herein do not inhibit the binding of C3bi to Mac-1 (shown herein by way of static adhesion assay and flow cytometry) is advantageous, because the host immune reaction is substantially uninhibited.

Overall, MAN-1 may not interfere with host defense mechanisms based on its activation-specific Mac-1 blockade and the selective epitope targeted by MAN-1.

Preferably the Mac-1 is present in a leukocyte or on the surface of a leukocyte. The adherence of leukocytes (e.g. monocyte, macrophages and neutrophils) is important in the inflammation process. Activation of neutrophils enables anchorage to the blood vessel endothelium, and increases responsiveness to chemotactic agents. Under the influence of C5a and leukotriene-B4, they exit from the circulation by migrating through gaps between endothelial cells, across the basement membrane and along the chemotactic gradient to the inflammation site. Leukocyte adhesion to the vessel wall or extracellular matrix is the basis of extravasation and transmigration of leukocytes at specific targets and thus plays a key role in various biological processes, such as inflammation. The ability of these adhesion molecules such as Mac-1 to react adequately on specific biological stimuli is a precondition for a regular function of these processes. This ability can be mediated either by quantitative changes in surface expression or by qualitative changes in receptor avidity or affinity. The latter is especially the case for the important group of integrins, of which Mac-1 is a member. These complex heterodimeric transmembrane receptors are characterized by the ability to become activated by performing a rapid conformational change and thereby changing the affinity for their natural ligands. This conformational change can be triggered by complex intracellular activation cascades leading to inside-out signaling.

The peptide, polypeptide or derivative of the invention may take a number of forms, however in a highly preferred form of the invention includes the amino acid sequence DSTLAPIFEY, DLWGFQLFDY, or DFWGSYDY. The skilled person will understand that once provided with the inventive sequences described supra, it will be possible to modify the residues and sequences to provide a peptide, polypeptide or derivative without totally destroying the ability to bind to activated Mac-1.

As mentioned, the invention includes derivatives of polypeptides described herein. As will be apparent below, once provided with the inventive amino acid sequences provided by the applicants, it will be possible to produce equivalent or derivative molecules that have the same or similar function to the specifically exemplified polypeptides.

For example, the skilled person will normally take the term “amino acid” to mean the natural (“D”) form of an amino acid. However, as used herein, the term “amino acid” and any reference to a specific amino acid is meant to include naturally occurring proteogenic amino acids as well as non-naturally occurring amino acids such as amino acid analogs. One of skill in the art understands that this definition includes, unless otherwise specifically indicated, naturally occurring proteogenic (D) or (L) amino acids, chemically modified amino acids, including amino acid analogs such as penicillamine (3-mercapto-D-valine), naturally occurring non-proteogenic amino acids such as norleucine and chemically synthesized compounds that have properties known in the art to be characteristic of an amino acid. As used herein, the term “proteogenic” indicates that the amino acid can be incorporated into a protein in a cell through well-known metabolic pathways.

The choice of including an (L)- or a (D)-amino acid into a peptide of the present invention depends, in part, on the desired characteristics of the peptide. For example, the incorporation of one or more (D)-amino acids can confer increasing stability on the peptide in vitro or in vivo. The incorporation of one or more (D)-amino acids also can increase or decrease the binding activity of the peptide as determined, for example, using the binding assays described herein, or other methods well known in the art. In some cases it is desirable to design a peptide that retains activity for a short period of time, for example, when designing a peptide to administer to a subject. In these cases, the incorporation of one or more (L)-amino acids in the peptide can allow endogenous peptidases in the subject to digest the peptide in vivo, thereby limiting the subject's exposure to an active peptide.

The invention also contemplates the use of amino acid equivalents. As used herein, the term “amino acid equivalent” refers to a compound, which departs from the structure of the naturally occurring amino acids, but which have substantially the structure of an amino acid, such that they can be substituted within a peptide, which retains is biological activity. Thus, for example, amino acid equivalents can include amino acids having side chain modifications or substitutions, and also include related organic acids, amides or the like. It will be understood that the term “residues” refers both to amino acids and amino acid equivalents.

The skilled artisan appreciates that limited modifications can be made to a peptide without destroying its biological function. Thus, modification of the peptides of the present invention that do not completely destroy their activity is within the definition of the compound claims as such. Specific types of genetically produced derivatives also include, but not limit by amino acid alterations such as deletions, substitutions, additions, and amino acid modifications. A “deletion” refers to the absence of one or more amino acid residue(s) in the related peptide. An “addition” refers to the presence of one or more amino acid residue(s) in the related peptide. Additions and deletions to a peptide may be at the amino terminus, the carboxy terminus, and/or internal, can be produced by mutation in polypeptide encoding DNA and/or by peptide post-translation modification.

Amino acid “modification” refers to the alteration of a naturally occurring amino acid to produce a non-naturally occurring amino acid. Analogs of polypeptides with unnatural amino acids can be created by site-specific incorporation of unnatural amino acids into polypeptides during the biosynthesis, as described in Christopher J. Noren, Spencer J. Anthony-Cahill, Michael C. Griffith, Peter G. Schultz, 1989 Science, 244:182-188.

A “substitution” refers to the replacement of one or more amino acid residue(s) by another amino acid residue(s) in the peptide. Mutations can be made in polypeptide encoding DNA such that a particular codon is changed to a codon, which codes for a different amino acid. Such a mutation is generally made by making the fewest nucleotide changes possible. A substitution mutation of this sort can be made to change an amino acid in the resulting peptide in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting peptide. To some extent the following groups contain amino acids which are interchangeable: the basic amino acids lysine, arginine, and histidine; the acidic amino acids aspartic and glutamic acids; the neutral polar amino acids serine, threonine, cysteine, glutarine, asparagine and, to a lesser extent, methionine; the nonpolar aliphatic amino acids glycine, alanine, valine, isoleucine, and leucine (however, because of size, glycine and alanine are more closely related and valine, isoleucine and leucine are more closely related); and the aromatic amino acids phenylalanine, tryptophan, and tyrosine. In addition, although classified in different categories, alanine, glycine, and serine seem to be interchangeable to some extent, and cysteine additionally fits into this group, or may be classified with the polar neutral amino acids. Although proline is a nonpolar neutral amino acid, its replacement represents difficulties because of its effects on conformation. Thus, substitutions by or for proline are not preferred, except when the same or similar conformational results can be obtained. The conformation conferring properties of proline residues may be obtained if one or more of these is substituted by hydroxyproline (Hyp). Derivatives can contain different combinations of alterations including more than one alteration and different types of alterations.

The ability of the derivative to retain some activity can be measured using techniques described herein and/or using techniques known to those skilled in the art for measuring the Mac-1 receptor-1 binding activity. “Derivatives” of peptides and polypeptides are functional equivalents having similar amino acid sequence and retaining, to some extent, the activities of the peptide or polypeptide. By “functional equivalent” is meant the derivative has an activity that can be substituted for the activity of the peptide or polypeptide. Preferred functional equivalents retain the full level of Mac-1 receptor-1-binding activity as measured by assays known to these skilled in the art, and/or in the assays described herein. Preferred functional equivalents have activities that are within 1% to 10,000% of the activity of the peptide or polypeptide, more preferably between 10% to 1000%, and more preferably within 50% to 200%. Derivatives have at least 50% sequence similarity, preferably 70%, mote preferably 90%, and even more preferably 95% sequence similarity to the peptide or polypeptide of the invention. “Sequence similarity” refers to “homology” observed between amino acid sequences in two different peptides or polypeptides, irrespective of origin.

In making modifications to the peptide, it will be appreciated that maintenance of secondary protein structure will assist in creating a molecule capable of binding to Mac-1. Various methods for constraining the secondary structure of a peptide are well known in the art. For example, peptides such as those containing -Phe-Pro-Gly-Phe- sequence form a S-turn, a well-known secondary structure. For example, a peptide can be stabilized by incorporating it into a sequence that forms a helix such as an alpha helix or a triple helix, according to methods described, for example, by Dedhar et al., (1987) J. Cell. Biol. 104:585; by Rhodes et al., (1978) Biochem 17:3442; and by Carbone et al., (1987) J. Immunol. 138:1838, each of which is incorporated herein by reference. Additionally, the peptides can be incorporated into larger linear, cyclic or branched peptides, so long as their receptor-binding activity is retained. The peptides of the present invention may be of any size so long as the Mac-1 receptor-binding activity is retained.

Another method for constraining the secondary structure of a newly synthesized linear peptide is to cyclize the peptide using any of various methods well known in the art. For example, a cyclized peptide of the present invention can be prepared by forming a peptide bond between non-adjacent amino acid residues as described, for example, by Schiller et al., (1985) Int. J. Pept. Prot. Res. 25:171, which is incorporated herein by reference. Peptides can be synthesized on the Merrifield resin by assembling the linear peptide chain using N-alpha-Fmoc-amino acids with Boc and tertiary-butyl side chain protection. Following the release of the peptide from the resin, a peptide bond can be formed between the amino and carboxy termini.

A newly synthesized linear peptide can also be cyclized by the formation of a bond between reactive amino acid side chains. For example, a peptide containing a cysteine-pair can be synthesized and a disulfide bridge can be formed by oxidizing a dilute aqueous solution of the peptide with K₃[Fe(CN)₆]. Alternatively, a lactam such as a glutamyl-lysine bond can be formed between lysine and glutamic acid residues, a lysinonorleucine bond can be formed between lysine and leucine residues or a dityrosine bond can be formed between two tyrosine residues. Cyclic peptides can be constructed to contain, for example, four lysine residues, which can form the heterocyclic structure of desmosine (see, for example, Devlin, Textbook of Biochemistry 3rd ed. (1992), which is herein incorporated by reference. Methods for forming these and other bonds are well known in the art and are based on well-known rules of chemical reactivity (Morrison and Boyd, Organic Chemistry, 6th Ed. (Prentice Hall, 1992), which is herein incorporated by reference).

The peptide, polypeptide or derivative of the present invention can be made by using well-known methods including recombinant methods and chemical synthesis. Recombinant methods of producing a peptide through the introduction of a vector including nucleic acid encoding the peptide into a suitable host cell is well known in the art, such as is described in Sambrook et al., Molecular Cloning: A Laboratory Manual, 2d Ed, Vols. 1 to 8, Cold Spring Harbor, N.Y. (1989), which is herein incorporated by reference. A linear sequence is synthesized, for example, by the solid phase peptide synthesis of Merrifield et al., J. Am. Chem. Soc., 85:2149 (1964), which is incorporated herein by reference). Alternatively, a peptide or polypeptide or derivative of the present invention can be synthesized using standard solution methods well known in the art (see, for example, Bodanszky, M., Principles of Peptide Synthesis (Springer-Veriag, 1984)), which is herein incorporated by reference). Newly synthesized peptides can be purified, for example, by high performance liquid chromatography (HPLC), and can be characterized using, for example, mass spectrometry or amino acid sequence analysis. Although a purity of greater than 95 percent for the synthesized peptide is preferred, lower purity may be acceptable. The analogs of the peptide or polypeptide can be peptides with altered sequence comprising another selection of L-alpha-amino acid residues, D-alpha-amino acid residues, non-alpha-amino acid residues.

The peptides, polypeptide or derivatives of the present invention may also be synthesized biologically. One example of a method of producing the peptide or polypeptide using recombinant DNA techniques entails the steps of (1) synthetically generating DNA oligonucleotide encoding peptide sequence, appropriated linkers and restriction sites coding sequences (2) inserting the DNA into an appropriate vector such as an expression vector, (3) inserting the gene containing vector into a microorganism or other expression system capable of expressing the inhibitor gene, and (7) isolating the recombinantly produced peptides.

Those skilled in the art will recognize that the peptides of the present invention may also be expressed in various cell systems, both prokaryotic and eukaryotic, ail of which are within the scope of the present invention. The appropriate vectors include viral, bacterial and eukaryotic expression vectors. A nucleic acid molecule, such as DNA, is said to be “capable of expressing” a peptide or polypeptide if it contains nucleotide sequences which contain transcriptional and translational regulatory information and such sequences are “operably linked” to nucleotide sequences which encode the peptide or polypeptide. The precise nature of the regulatory regions needed for gene sequence expression may vary from organism to organism, but shall in general include a promoter region which, in prokaryotes, contains both the promoter (which directs the initiation of RNA transcription) as well as the DNA sequences which, when transcribed into RNA, will signal synthesis initiation. Such regions will normally include those 5′-non-coding sequences involved with initiation of transcription and translation, such as the TATA box, capping sequence, CAAT sequence, and the like.

For example, the entire coding sequence of the polypeptide may be combined with one or more of the following in an appropriate expression vector to allow for such expression: (1) an exogenous promoter sequence (2) a ribosome binding site (3) carrier protein (4) a polyadenylation signal (4) a secretion signal. Modifications can be made in the 5′-untranslated and 3′-untranslated sequences to improve expression in a prokaryotic or eukaryotic cell; or codons may be modified such that while they encode an identical amino acid, that codon may be a preferred codon in the chosen expression system, The use of such preferred codons is described in, for example, Grantham et al., (1981) Nuc. Acids Res., 9:43-74 and Lathe, (1985) J. Mol. Biol., 183:1-12, hereby incorporated by reference herein in their entirety. Moreover, once cloned into an appropriate vector, the DNA can be altered in numerous ways as described above to produce functionally equivalent variants thereof.

In another embodiment, the peptides or polypeptides of present invention can be expressed as fusion proteins fused at the N-terminus or C-terminus, or at both termini, to one or more of peptides or polypeptides. In a preferred embodiment, the fusion protein is specifically cleavable such that at least a substantial portion of the peptide sequence can be proteolytically cleaved away from the fusion protein to yield the desired polypeptide. The fusion proteins of the invention can be designed with cleavage sites recognized by chemical or enzymatic proteases. In one embodiment, the fusion protein is designed with a unique cleavage site (or sites) for removal of the polypeptide sequence, i.e. the fusion protein is designed such that a given protease (or proteases) cleaves away the polypeptide sequence but does not cleave at any site within the sequence of the desired protein, avoiding fragmentation of the desired protein. In another embodiment, the cleavage site (or sites) at the fusion joint (or joints) is designed such that cleavage of the fusion protein with a given enzyme liberates the authentic, intact sequence of the desired protein from the remainder of the fusion protein sequence. The pTrcHisA vector (Invitrogen) and other like can be used to obtain high-level, regulated transcription from the trc promoter for enhanced translation efficiency of fusion protein in E. coli. The peptides or polypeptides or polypeptides of the invention can be expressed fused to an N-terminal nickel-binding poly-histidine tail for one-step purification using metal affinity resins. The enterokinase cleavage recognition site in the fusion protein allows for subsequent removal of the N-terminal histidine fusion protein from the purified recombinant protein. The polypeptide fusion protein can be produced using appropriated carrier protein, for example, .beta.-galactosidase, green fluorescent protein, luciferase, dehydrofolate reductase, thireodoxin, protein A Staphylococcus aureus and glutathione S-transferase. These examples are, of course, intended to be illustrative rather than limiting.

The peptides or polypeptides of present invention can be synthesized as a fusion protein with a virus coat protein and expressed on the surface of virus particle, for example bacteriophage M13, T7, T4 and lambda, lambda gt10, lambda gt11 and the like; adenovirus, retrovirus and pMAM-neo, pKRC and the like.

In general, prokaryote expression vectors contain replication and control sequences, which are derived from species compatible with the host cell. The vector ordinarily carries a replication site, as well as sequences that encode proteins capable of providing phenotypic selection in transformed cells. For example, vectors include pBR322 (ATCC No. 37,017), phGH107 (ATCC No. 40,011), pBO475, pS0132, pRIT5, any vector in the pRIT20 or pRIT30 series (Nilsson and Abrahmsen, Meth. Enzymol., 185: 144-161 (1990)), pRIT2T, pKK233-2, pDR540, pPL-lambda, pQE70, pQE60, pQE-9 (Qiagen), pBS, phagescript, psiX174, pBluescript SK, pBsKS, pNH8a, pNH16a, pNH18a, pNH46a (Stritagene); pTRC99A, pKK223-3, pKK233-3, pDR540, pRIT5 (Pharmacia). Eukaryotic: pWLneo, pSV2cat, pOG44, pXT1, pSG (Stratagene) pSVK3, PBPV, pMSG, PSVL (Pharmacia) are suitable for expression in prokaryotic hosts. Such plasmids are, for example, disclosed by Sambrook (cf. “Molecular Cloning: A Laboratory Manual”, second edition, edited by Sambrook, Fritsch, & Maniatis, Cold Spring Harbor Laboratory, (1989)). Bacillus plasmids include pC194, pC221, pT127, and the like. Such plasmids are disclosed by Gryczan (In: The Molecular Biology of the Bacilli, Academic Press, NY (1982), pp. 307-329). Suitable Streptomyces plasmids include p1J101 (Kendall et al., (1987) J. Bacteriol. 169 4177-4183, and streptomyces bacteriophages such as .phi.C31 (Chater et al., In: Sixth International Symposium on Actinomycet ales Biology, Akademiai Kaido, Budapest, Hungary (1986), pp. 45-54). Pseudomonas plasmids are reviewed by John et al. ((1986) Rev. Infect. Dis. 8:693-704), and Izaki ((1978) Jpn. J. Bacteriol. 33:729-742).

Prokaryotic host cells containing the expression vectors of the present invention include E. coli K12 strain 294 (ATCC NO 31446), E. coli strain JMIO (Messing et al., Nucl. Acid Res., 9: 309 (1981)), E. coli strain B, E. coli strain chi 1776 (ATCC No. 31537), E. coli c600 (Appleyard, (1954) Genetics, 39:440), E. coli W3110 (F-, .gamma-, prototrophic, ATCC No. 27325), E. coli strain 27C7 (W3110, tonA, phoA E15, (argF-lac)169, ptr3, degP41, ompT, kan^{r}) (U.S. Pat. No. 5,288,931, ATCC No. 55,244), Bacillus subtilis, Salmonella typhimurium, Serratia marcesans and Pseudomonas species. For example, E. coli K12 strain MM 294 (ATCC No. 31,446) is particularly useful. Other microbial strains that may be used include E. coli strains such as E. coli B and E. coli X1776 (ATCC No. 31,537). These examples are, of course, intended to be illustrative rather than limiting.

To express of peptides or polypeptides of the invention (or a functional derivative thereof) in a prokaryotic cell, it is necessary to operably link the peptide-encoding sequence to a functional prokaryotic promoter. Such promoters may be either constitutive or, more preferably, regulatable (i.e., inducible or derepressible). Examples of constitutive promoters include the int promoter of bacteriophage lambda., the bla promoter of the .beta.-lactamase gene sequence of pBR322, and the CAT promoter of the chloramphenicol acetyl transferase gene sequence of pPR325, and the like. Examples of inducible prokaryotic promoters include the major right and left promoters of bacteriophage lambda, the trp, recA, .lambda.acZ, .lambda.acl, and gal promoters of E. coli, the .alpha.-amylase (Ulmanen et al., (1985) J. Bacteriol. 162:176-182) and the .zeta.-28-specific promoters of B. subtilis (Gilman et al., (1984) Gene sequence 32:11-20), the promoters of the bacteriophages of Bacillus (Gryczan, In: The Molecular Biology of the Bacilli, Academic Press, Inc., NY (1982)), and Streptomyces promoters (Ward et al., (1986) Mol. Gen. Genet. 203:468-478). The most commonly used in recombinant DNA construction promoters include the β-lactamase (penicillinase) and lactose promoter systems (Chang et al., (1978) Nature, 375:615; Itakura et al., (1977) Science, 198, 1056; Goeddel et al., (1979) Nature, 281, 544) and a tryptophan (trp) promoter system (Goeddel et al., (1980) Nucleic Acids Res., 8:4057; EPO Appl. Publ. No. 0036,776). While these are the most commonly used, other microbial promoters have been discovered and utilized, and details concerning their nucleotide sequences have been published, enabling a skilled worker to ligate them functionally with plasmid vectors (see, e.g., Siebenlist et al. (1980) Cell, 20, 269.

Proper expression in a prokaryotic cell also requires the presence of a ribosome binding site upstream of the gene sequence-encoding sequence. Such ribosome binding sites are disclosed, for example, by Gold et al. (1981) Ann. Rev. Microbiol. 35:365-404). The ribosome binding site and other sequences required for translation initiation are operably linked to the nucleic acid molecule encoding peptides or polypeptides of the invention. Translation in bacterial system is initiated at the codon with encode the first methionine. For this reason, it is preferable to include the ATG codon in peptide sequence and to ensure that the linkage between a prormoter and a DNA sequence that encodes a peptide does not contain any intervening codons that are capable of encoding a methionine.

In addition to prokaryotes, eukaryotic organisms, such as yeast, or cells derived from multicellular organisms can be used as host cells. Saccharomyces cerevisiae, or common baker's yeast, is the most commonly used among eukaryotic microorganisms, although a number of other strains are commonly available. For expression in Saccharomyces, the plasmid YRp7, for example (Stinchcomb et al., (1979) Nature 282 39; Kingsman et al., (1979) Gene 7:141; Tschemper et al., (1980) Gene 10:157), is commonly used. This plasmid already contains the trp1 gene that provides a selection marker for a mutant strain of yeast lacking the ability to grow in tryptophan, for example, ATCC No. 44,076 or PEP4-1 (Jones, (1977) Genetics, 85, 12). The presence of the trpl lesion as a characteristic of the yeast host cell genome then provides an effective environment for detecting transformation by growth in the absence of tryptophan. Suitable promoting sequences in yeast vectors include the promoters for 3-hosphoglycerate kinase (Hitzeman et al., (1980) J. Biol. Chem. 255:2073) or other glycolytic enzymes (Hess et al., (1968) J. Adv. Enzyme Reg. 7:149; Holland et al., (1978) Biochemistry 17:4900), such as enolase, glyceraldehyde-3-phosphate dehydrogenase, hexokinase, pyruvate decarboxylase, phosphofructokinase, glucose-6phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are also ligated into the expression vector 3′ of the sequence desired to be expressed to provide polyadenylation of the mRNA and termination. Other promoters, which have the additional advantage of transcription controlled by growth conditions, are the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization. Any plasmid vector containing yeast-compatible promoter, origin of replication and termination sequences is suitable.

In addition, plant cells are also available as hosts, and control sequences compatible with plant cells are available, such as the cauliflower mosaic virus 35S and 19S, and nopaline synthase promoter and polyadenylation signal sequences. Another preferred host is an insect cell, for example the Drosophila larvae. Using insect cells as hosts, the Drosophila alcohol dehydrogenase promoter can be used. Rubin, (1988) Science 240:1453-1459.

However, peptides or polypeptides of present invention can be expressed in vertebrata host cells. The propagation of vertebrate cells in culture (tissue culture) has become a routine procedure in recent years (Tissue Culture, Academic Press, Knise and Patterson, editors (1973). Examples of useful mammalian host cells include monkey kidney CV1 line transformed by SV40 (COS-7, ATCC CRL 1651); human embryonic kidney line (293 or 293 cells subcloned for growth in suspension culture, Graham et al., (1977) J. Gen Virol., 36: 59); baby hamster kidney cells (BHK, ATCC CCL 10); Chinese hamster ovary cells/−DHFR (CHO, Urlaub and Chasin, (1980) Proc. Nad. Acad. Scl. USA, 77:4216); mouse sertoli cells (TM4, Mather, (1980) Biol. Reprod., 23: 243-251); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., (1982) Annals N.Y. Acad. Sci, 383: 44-68); MRC 5 cells; FS4 cells; and a human hepatoma cell line (Hep G2). For expression in mammalian host cells, useful vectors include, but not limited vectors derived from SV40, vectors derived from cytomegalovirus such as the pRK vectors, including pRK5 and pRK7 (Suva et al., (1987) Science, 237:893-896, EP 307,247 (Mar. 15, 1989), EP 278,776 (Aug. 17, 1988)) vectors derived from vaccinia viruses or other pox viruses, and retroviral vectors such as vectors derived from Moloney's murine leukemia virus (MoMLV).

The expression of peptides or polypeptides of the invention in eukaryotic hosts requires the use of eukaryotic regulatory regions. Such regions will, in general, include a promoter region sufficient to direct the initiation of RNA synthesis. Preferred eukaryotic promoters include, for example, the promoter of the mouse net allothionein I gene sequence (Hamer et al., (1982) J. Mol. Appl. Gen. 1:273-288); the TK promoter of Herpes virus (McKnight, (1982) Cell 31:355-365); the SV40 early promoter (Benoist et al., (1981) Nature (London) 290:304-310); the yeast gal4 gene sequence promoter (Johnston et, (1982) Proc. Natl. Acad. Sci. (USA) 79:6971-6975; Silveret al., (1984) Proc. Natl. Acad Sci (USA) 81:5951-5955). An origin of replication may be provided either by construction of the vector to include an exogenous origin, such as may be derived from SV40 or other viral (e.g., Polyoma, Adeno, VSV, BPV) source, or may be provided by the host cell chromosomal replication mechanism. If the vector is integrated into the host cell chromosome, the latter is often sufficient. Satisfactory amounts of protein are produced by cell cultures; however, refinements, using a secondary coding sequence, serve to enhance production levels even further. One secondary coding sequence comprises dihydrofolate reductase (DHFR that is affected by an externally controlled parameter, such as methotrexate (MTX), thus permitting control of expression by control of the methotrexate concentration (Urlaub and Chasin, (1980) Proc. Natl. Acad, Sci. (USA) 77, 4216).

Optionally, the DNA encoding peptides or polypeptides of the invention is operably linked to a secretory leader sequence resulting in secretion of the expression product by the host cell into the culture medium. Examples of secretory leader sequences include stII, ecotin, lamB, herpes GD, Ipp, alkaline phsophatase, invertase, and alpha factor. Also suitable for use herein is the 36 amino acid leader sequence of protein A (Abrahmsen et al., (1985) EMBO J., 4: 3901).

Once the vector or nucleic acid molecule containing the construct(s) has been prepared for expression, the DNA construct(s) may be introduced into an appropriate host cell by any of a variety of suitable means, i.e., transformation, transfection, conjugation, protoplast fusion, electroporation, particle gun technology, lipofection, calcium phosphate precipitation, direct microinjection, DEAE-dextran transfection, and the like. The most effective method for transfection of eukaryotic cell lines with plasmid DNA varies with the given cell type. After the introduction of the vector; recipient cells are grown in a selective medium, which selects for the growth of vector-containing cells. Expression of the cloned gene molecule(s) results in the production of peptides or polypeptides of the invention. This can take place in the transformed cells as such, or following the induction of these cells to differentiate (for example, by administration of bromodeoxyuracil to neuroblastoma cells or the like). A variety of incubation conditions can be used to form the peptide of the present invention. The most preferred conditions are those which mimic physiological conditions.

Transfection refers to the taking up of an expression vector by a host cell whether or not any coding sequences are in fact expressed. Numerous methods of transfection are known to the ordinarily skilled artisan, for example, CaPO₄.precipitation and electroporation. Successful transfection is generally recognized when any indication of the operation of this vector occurs within the host cell.

Transformation means introducing DNA into an organism so that the DNA is replicable, either as an extrachromosomal element or by chromosomal integrant. Depending on the host cell used, transformation is done using standard techniques appropriate to such cells. The calcium treatment employing calcium chloride, as described in section 1.82 of Sambrook et al., Molecular Cloning (2nd ed.), Cold Spring Harbor Laboratory, New York (1989), is generally used for prokaryotes or other cells that contain substantial cell-wall barriers. Infection with Agrobacterium tumefaciens is used for transformation of certain plant cells, as described by Shaw et al., (1983) Gene, 23: 315 and WO 89/05859 published Jun. 29, 1989, For mammalian cells without such cell walls, the calcium phosphate precipitation method described in sections 16.30-16.37 of Sambrook et al., supra, is preferred. General aspects of mammalian cell host system transformations have been described by Axel in U.S. Pat. No. 4,399,216 issued Aug. 16, 1983. Transformations into yeast are typically carried out according to the method of Van Solingen et al., J. Bact., 130: 946 (1977) and Hsiao et al., Proc. Natl. Acad. Sci. (USA), 76: 3829 (1979). However, other methods for introducing DNA into cells such as by nuclear injection, electroporation, or by protoplast fusion may also be used.

The host cells used to produce the peptides or polypeptides of the invention can be cultured in a variety of media, as described generally in Sambrook et al. A wide variety of transcriptional and translational regulatory sequences may be employed, depending upon the nature of the host to control the expression. Transcriptional initiation regulatory signals may be selected which allow for repression or activation, so that expression of the gene sequences can be modulated. Of interest are regulatory signals, which are temperature-sensitive so that by varying the temperature, expression can be repressed or initiated, or are subject to chemical (such as metabolite) regulation.

In an intracellular expression system or periplasmic space secretion system, the recombinantly expressed peptides or polypeptides of the invention can be recovered from the culture cells by disrupting the host cell membrane/cell wall (e.g., by osmotic shock or solubilizing the host cell membrane in detergent). Alternatively, in an extracellular secretion system, the recombinant peptide can be recovered from the culture medium. As a first step, the culture medium or lysate is centrifuged to remove any particulate cell debris. The membrane and soluble protein fractions are then separated. The Z domain variant peptide can then be purified from the soluble protein fraction. If the peptide is expressed as a membrane bound species, the membrane bound peptide can be recovered from the membrane fraction by solubilization with detergents. The crude peptide extract can then be further purified by suitable procedures such as fractionation on immunoaffinity or ion-exchange columns; ethanol precipitation; reverse phase HFLC; chromatography on silica or on a cation exchange resin such as DEAE; chromatofocusing; SDS-PAGE; ammonium sulfate precipitation; gel filtration using, for example, Sephadex G-75; hydrophobic affinity resins and ligand affinity using IgG ligand immobilized on a matrix. In vitro transcription/translation systems can also be employed to produce peptides or polypeptides of the present invention using RNAs derived from the polypeptide encoding DNA constructs. Cell-free translation systems have been used in the biosynthesis of proteins and peptides, and have become a standard tool in molecular biology for protein production (in vitro transcription and translation protocols, Methods in Molecular Biology, 37 Edited by M. J. Tymms, 1995, Humana Press. Inc., Merrick, Translation of exogenous mRNAs in reticulocyte lysates, Meth. Enzymol. 101:38 (1983)). Kigawa, T. and Yokohama, S., “Continuous Cell-Free Protein Synthesis System for Coupled Transcription-Translation” Journal of Biochemistry 110:166-168 (1991), Baranov et al., “Gene expression in a cell-free system on the preparative scale” (1989) Gene 84:463-466, Kawarasaki et al., “A long-lived batch reaction system of cell-free protein synthesis” (1995) Analytical Biochemistry 226:320-324). Both eukaryotic and prokaryotic cell-free systems can be used for in vitro polypeptide synthesis. The rabbit reticulocyte (Pelham and Jackson, (1976) Eur. J. Biochem., 67: 247-256) and wheat germ lysate (Roberts and Paterson, (1973) Proc. Natl. Acad. Sci, 70: 2330-2334) methods are commonly used eukaryotic in vitro translation systems. The E. coli S30 extract method devised by Spirin, A. S. et al., “Continuous Cell-Free Translation System Capable of Producing Polypeptides in High Yield” (1988) Science 242 (4882):1162-1164, Zubay, (1973) Ann. Rev. Genet., 7:267, and the fractionated method of Gold and Schweiger, (1971) Meth. Enzymol., 20: 537 are widely used prokaryotic in vitro translation systems.

The expression unit for in vitro synthesis comprises a 5′ untranslated region and may additionally comprise a 3′ region. The 5′ untranslated region of the expression unit contains a promoter or RNA polymerase binding sequence, a ribosome binding sequence, and a translation initiation signal. The 5′ untranslated region (“head”) may also contain convenient restriction sites and a translation enhancer or “Activator” sequence(s). The 3′ region may contain convenient restriction sites and a 3′ tail of a selected sequence. The expression unit may be chemically synthesized by protocols well known to those skilled in the art. Alternatively, these elements may be incorporated into one or more plasmids, amplified in microorganisms, purified by standard procedures, and cut into appropriate fragments with restriction enzymes before assembly into the expression unit.

The 5′ untranslated region contains a promoter or RNA polymerase binding sequence, such as those for the T₇, T₃, or SP6 RNA polymerase. Positioned downstream of or within the promoter region is a DNA sequence, which codes for a ribosomal binding site. This ribosome binding site may be specific for prokaryotic ribosomal complexes (including ribosomal RNAs) if a prokaryotic translation procedure is used. However, a preferred embodiment of this invention uses a eukaryotic sequence and an in vitro eukaryotic translation system, such as the rabbit reticulocyte system (Krawetz et al., 1983 Can. J. Biochem. Cell. Biol. 61:274-286; Merrick, 1983 Meth. Enzymol. 101:38). A consensus translation initiation sequence, GCCGCCACCATGG as well as other functionally related sequences have been established for vertebrate mRNAs (Kozak, 1987 Nucleic Acids Res, 15:8125-8148). This sequence or related sequences may be used in the DNA construction to direct protein synthesis in vitro. The ATG triplet in this initiation sequence is the translation initiation codon for methionine; in vitro protein synthesis is expected to begin at this point.

Between the promoter and translation initiation site, it may be desirable to place other known sequences, such as translation enhancer or “activator” sequences. For example, Jobling et al. (1988 Nucleic Acids Res. 16:4483-4498) showed that the untranslated “leader sequences” from tobacco mosaic virus “stimulated translation significantly” in SP6-generated mRNAs. They also reported that the 36-nucleotide 5′ untranslated region of alfalfa mosaic virus RNA 4 increases the translational efficiency of barley amylase and human interleukin mRNAs (Jobling and Gehrke, 1987 Nature 325:622-625). Black beetle virus (Nodavirus) RNA 2 (Friesen and Rueckert, J. 1981 Virol. 37:876-886), turnip mosaic virus, and brome mosaic virus coat protein mRNAs (Zagorski et al., Biochimie 65:127-133, 1983) also translate at high efficiencies. In contrast, certain untranslated leaders severely reduce the expression of the SP6 RNAs (Jobling et al. (1988 Nucleic Acids Res. 16:4483-4498).

In addition, polypeptide encoding DNA may be incorporated into the in vitro expression unit. In one embodiment, the expressed polypeptides contain both carrier polypeptide/peptide and the polypeptide of the invention. The carrier peptide would be useful for quantifying the amount of fusion polypeptide and for purification (given that an antibody against the carrier polypeptide is available or can be produced). One example is 6His amino acid sequence; the second is the 11 amino acid Substance P, which can be attached as fusion peptides to peptides of the invention. Anti-6 His and anti-Substance P antibodies are commercially available for detecting and quantifying fusion proteins. Another example is the eight amino acid marker peptide, “Flag” (Hopp et al., 1988 Bio/Technology 6:1204-1210). A preferred form of the carrier polypeptide is one which may be cleaved from the novel polypeptide by simple chemical or enzymatic means.

In a further preferred form of the invention the polypeptide or derivative is in the form of a single-chain antibody molecule. Recent advances in antibody engineering have allowed the genes encoding antibodies to be manipulated, so that antigen binding molecules can be expressed within mammalian cells in a controlled way. Application of gene technologies to antibody engineering has enabled the synthesis of single-chain fragment variable (scFv) antibodies that combine within a single polypeptide chain the light and heavy chain variable domains of an antibody molecule covalently joined by a predesigned peptide linker. The resultant scFv gene can be expressed in bacterial expression systems such as E. coli. Bundled in the “gene display package” single-chain antibodies displayed at the surface of filamentous phages of the M13 family provided the possibility to create antibody libraries both from various living sources and products of diversification of a single scFv molecule. Antibodies with the desired specificity can be isolated from such libraries employing effective selection techniques (panning) in which the antigen is immobilized on a solid support.

Thus, a further preferred form of the polypeptide is a single chain antibody including an amino acid sequence motif as described herein. In a more highly preferred form of the invention, the single chain antibody includes one or more of the following regions HCDR1, HCDR2, HCDR3, LINKER, LCDR1, LCDR2, LCDR3.

In one embodiment, the HCDR1 is MSGFIFRDYDMD or MSGFSNYGIH or equivalent sequence, the HCDR2 is independently TSSYTIQDAA or VALISYDNGNKKFYA or equivalent sequence, the HCDR3 region is independently DLWGFQLFDY, DFWGSYDY or DSTLAPIFEY or equivalent sequence, the LINKER is independently KLEEGEFSEARV or equivalent sequence, the LCDR1 is independently GGNNIGSKSVH or GGNNIGSTTVH or equivalent sequence, the LCDR2 is independently YDSVRPS or DDNERPS or equivalent sequence, the LCDR3 is independently QVWDSNTDHYV or QVWDSGSDHW or equivalent sequence.

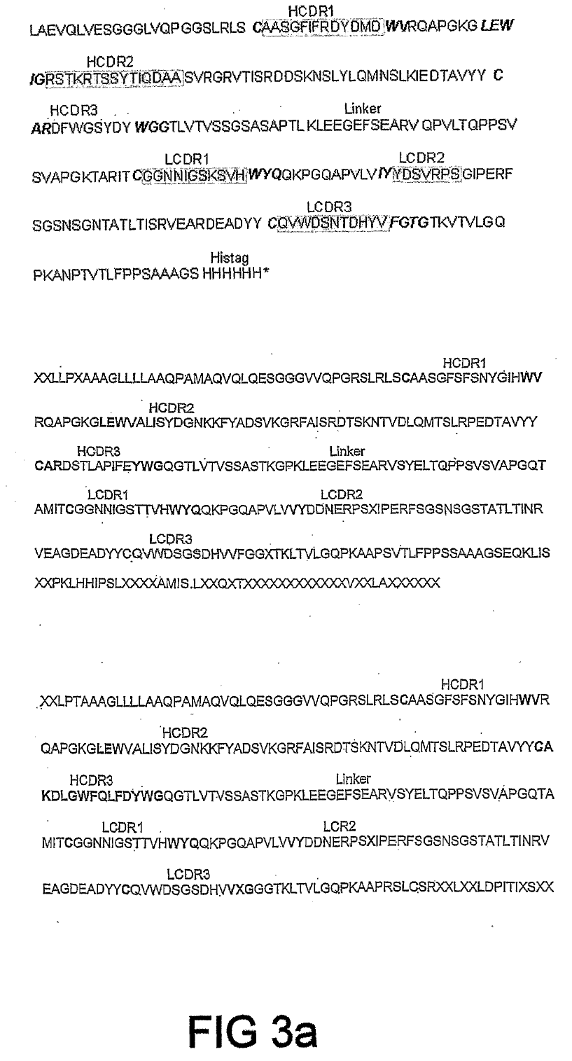

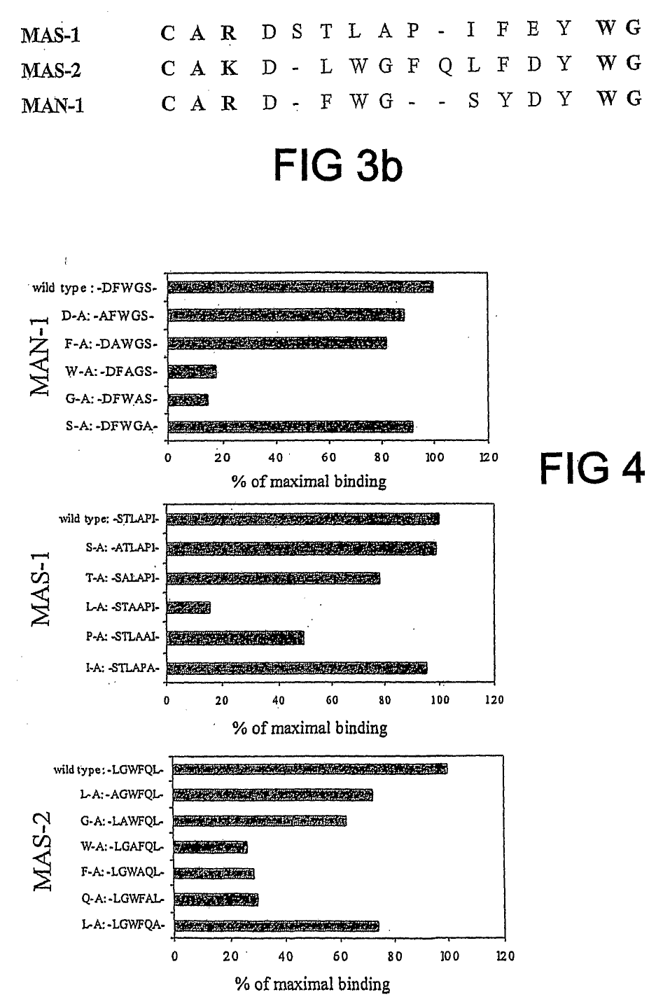

The amino acid sequences of three single chain antibodies of the present invention are shown in FIG. 3a. These sequences contain His tag regions, and it will be understood that these regions are present to facilitate affinity purification of the molecules. Once in possession of the full sequences of three exemplary single chain antibodies, the skilled person could truncate the sequence or add further residues in order to provide other antibody molecules useful in the context of the invention. Various derivatives could also be produced as discussed elsewhere herein. It would be a matter of routine for the skilled artisan to manipulate the exemplary sequences and test whether the altered molecule has an ability to bind activated Mac-1.

Single chain antibodies can be produced in various hosts, including bacteria (e.g. E. coli), yeast (e.g. Pichia Pastoris, S. cerevisae), mammalian and insect cell cultures (CHO cells, baculovirus expression systems, and others), transfected or transgenic plants and animals (such as rice, tobacco, potatoes, cows, or goats).

These molecules are amenable to protein modifications, such as: site-directed PEGylation and glycosylation Oligo- and multimerization. Single chain antibodies are also amenable to protein engineering, including conjugation and fusion to other proteins, advantageous expression, higher stability and solubility designs, reduction of immunogenicity, for example by humanization and/or de-immunization

Single chain antibodies used as therapeutics provide high tissue penetration, fast clearance (often useful for high tumor to healthy tissue ratio and certain acute-care applications), renal clearance depending on their engineered size (avoiding potential dose limiting effects that otherwise might come from hepatotoxicity), and no intrinsic effector function thereby limiting potential immunogenicity issues.

The single chain antibody format allows the genetic fusion of effector molecules such that particular effector molecules can be targeted to a site in the body exhibiting inflammation. In this way, the potentially toxic effects of effector molecules (eg cytotoxic drugs) can be sequestrated away from the systemic circulation, and localized to the area of greatest need. Details of coupling techniques and various effector molecules are described elsewhere herein.

Given the biological activity of the polypeptide or derivatives described herein, the present invention will be useful in methods of medical treatment. Mac-1 has been implicated in many pathophysiological states such as inflammation. Accordingly, the present invention provides a composition including a polypeptide or derivative as described herein in and a pharmaceutically acceptable carrier. The composition is contemplated to have use in the treatment of a condition selected from the group consisting of Crohn's disease, collitis ulcerosa, multiple sclerosis, sarcoidosis, psoriasis, atherosclerosis and its clinical sequelae, scleroderma, intestinal adhesions, hypertrophic scars, rheumatoid arthritis, septicemia, autoimmune disease, acute coronary syndrome, HIV infection, reperfusion injuries, ischemia, neointimal thickening, infiltration of polymorpholeucocytes, autoimmune disease, and neovascularisation-mediated diseases. Other diseases and conditions not detailed herein may benefit from the present invention, and it will be a matter of routine experimentation to identify further medical uses.

The administration of therapeutic peptides or polypeptides can be performed in many ways. However, given the presence of acids and proteases in the gastrointestinal tract, peptides and polypeptides are generally administered by IV, IM, subcutaneous, or topical routes. A major difficulty with the delivery of therapeutic proteins is their short plasma half-life, mainly due to rapid serum clearance and proteolytic degradation via the action of peptidases. Peptidases break a peptide bond in peptides by inserting a water molecule across the bond. Generally, most peptides are broken down by peptidases in the body in a manner of a few minutes or less. In addition, some peptidases are specific for certain types of peptides, making their degradation even more rapid. Thus, if a peptide is used as a therapeutic agent, its activity may be generally reduced if the peptide degrades in the body due to the action of peptidases. One way to overcome this disadvantage is to administer large dosages of the therapeutic peptide of interest to the patient so that even if some of the peptide is degraded, enough remains to be therapeutically effective.

Another possibility is to block the action of peptidases to prevent degradation of the therapeutic peptide or to modify the therapeutic peptides and polypeptides in such a way that their degradation is slowed down while still maintaining biological activity. Such methods include conjugation with polymeric materials such as dextrans, polyvinyl pyrrolidones, glycopeptides, polyethylene glycol and polyamino acids, conjugation with adroitin sulfates, as well as conjugation with polysaccharides, low molecular weight compounds such as aminolethicin, fatty acids, vitamin B₁₂, and glycosides.

Peptide therapeutics may also be delivered topically. For example, non-ionic liposomal systems have been shown to be useful in delivering therapeutic amounts of growth hormone releasing peptide across the skin. Similarly, peptides useful in the treatment of psoriasis have been successfully delivered using Novasome® technology. Novasome microvesicles are paucilamellar vesicles that can be formed from many bio-compatible, single-tailed amphiphiles, as well as phopholipids. Novasome microvesicles have up to seven bilayer membranes, each composed of these amphiphilic molecules, surrounding a large amorphous core. The core accounts for most of the Novasome vesicle volume, providing a high capacity for water soluble and water immiscible substances, as well as some small solid particles. Because of these unique traits, Novasome microvesicles have many advantages over conventional liposomes.

Peptides and polypeptides may also be delivered across other non-dermal structures, such as mucous membranes. Accordingly, the present invention contemplates the delivery of the inventive polypeptide or derivatives via the buccal route, sublingual route, rectal route, intrathecal route, vaginal route, nasal route, ocular route, and pulmonary route.

The present invention also provides analogs of the pharmaceutical composition which can comprise in its molecular structure residues being derivatives of compounds other than amino acids, referenced herein as “peptide mimetics” or “peptidomimetics” (Fauchere, J. (1986) Adv. Drug Res. 15: 29; Veber and Freidinger (1985) TINS p. 392; and Evans et al. (1987) J. Med. Chem. 30: 1229, which are incorporated herein by reference) and can be developed, for example, with the aid of computerized molecular modeling.

Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biochemical property or pharmacological activity), but have one or more peptide linkages optionally replaced by a linkage selected from the group consisting of: —CH₂—NH—, —CH₂S—, —CH₂—CH₂—, —CH═CH— (cis and trans), —COCH₂—, —CH(OH)CH₂—, and —CH₂ SO—, by methods known in the art and further described in the following references: Spatola, A. F. in “Chemistry and Biochemistry of Amino Acids, Peptides, and Proteins,” B. Weinstein, eds., Marcel Dekker, New York, p. 267 (1983); Spatola, A. F., Vega Data (March 1983), Vol. 1, Issue 3, “Peptide Backbone Modifications” (general review); Morley, J. S., Trends Pharm Sci (1980) pp. 463-468 (general review); Hudson, D et al., (1979) Int J Pept Prot Re 14:177-185 (—CH₂NH—, —CH₂—CH₂—); Spatola, A. F. et al., (1986) Life Sci 38:1243-1249 (—CH₂—S); Hann, M. M., (1982) J Chem Soc Perkin Trans 1 307-314 (—CH═CH—, cis and trans); Almquist, R. G. et al., (1980) J Med Chem 23: 1392-1398 (—COCH₂—); Jennings-White, C. et al., (1982) Tetrahedron Lett 23:2533 (—COCH₂—); Szelke, M. et al., European Appln. EP 45665 (1982) CA: 97:39405 (1982) (—CH(OH)CH₂—); Holladay, M. W. et al., (1983) Tetrahedron Lett 24:4401-4404 (—C(OH)CH₂—); and Hruby, V. J., (1982) Life Sci 31:189-199 (—CH₂—S—); each of which is incorporated herein by reference.

In another embodiment, a particularly preferred non-peptide linkage is —CH₂ NH—. Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others.

Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macromolecules(s) to which the peptidomimetic binds to produce the therapeutic effect. Derivitization (e.g., labelling) of peptidomimetics should not substantially interfere with the desired biological or pharmacological activity of the peptidomimetic.

A variety of designs for peptide mimetics are possible. For example, cyclic peptides, in which the necessary conformation for binding is stabilized by nonpeptides, are specifically contemplated. U.S. Pat. No. 5,192,746 to Lobl, et al., U.S. Pat. No. 5,169,862 to Burke, Jr., et al, U.S. Pat. No. 5,539,085 to Bischoff, et al., U.S. Pat. No. 5,576,423 to Aversa, et al., U.S. Pat. No. 5,051,448 to Shashoua, and U.S. Pat. No. 5,559,103 to Gaeta, et al., all hereby incorporated by reference, describe multiple methods for creating such compounds. Synthesis of nonpeptide compounds that mimic peptide sequences is also known in the art. Eldred, et. al., (J. Med. Chem. 37:3882 (1994)) describe nonpeptide antagonists that mimic the peptide sequence.

A variety of carriers can be associated with the polypeptide including, but not limited to synthetic, semi-synthetic and natural compounds such as polypeptides, lipids, carbohydrates, polyamines, synthetic polymers, that form solutions (unimolecular systems), dispersions (supramolecular systems), or any particular systems such as nanoparticles, microspheres, matrixes, gels and other.

Therefore, in one embodiment, this invention provides a pharmaceutical composition comprising at least one polypeptide or derivative thereof, wherein said polypeptide or derivative thereof is capable of specific binding with the high affinity Mac-1 receptor-1 or a derivative of the Mac-1 receptor-1 and structural similar receptors further comprises a carrier.

The following classes of carriers are given as examples. It is understood, however, that a variety of other carriers can be used in the present invention.

The polymeric carriers can be nonionic water-soluble, nonionic hydrophobic or poorly water soluble, cationic, anionic or polyampholite, such as a polypeptides.

Preferred hydrophilic carrier is a nontoxic and non-immunogenic polymer which is soluble in water, Such segments include (but not are limited to) polyethers (e.g., polyethylene oxide), polysaccharides (e.g., dextran), polyglycerol, homopolymers and copolymers of vinyl monomers (e.g., polyacrylamide, polyacrylic esters (e.g., polyacryloylmorpholine), polymethacrylamide, poly(N-(2 hydroxypropyl)methacrylamide, polyvinyl alcohol, polyvinyl pyrrolidone, polyvinyltriazole, N-oxide of polyvinylpyridine, copolymer of vinylpyridine and vinylpyridine N-oxide) polyortho esters, polyaminoacids, polyglycerols (e.g., poly-2-methyl-2-oxazoline, poly-2-ethyl-2-oxazoline) and copolymers and derivatives thereof.

Preferred nonionic hydrophobic and poorly water soluble segments include polypropylene oxide, copolymers of polyethylene oxide and polyethylene oxide, polyalkylene oxide other than polyethylene oxide and polypropylene oxide, homopolymers and copolymers of styrene (e.g., polystyrene), homopolymers and copolymers isoprene (e.g., polyisoprene), homopolymers and copolymers butadiene (e.g., polybutadiene), homopolymers and copolymers propylene (e.g., polypropylene), homopolymers and copolymers ethylene (e.g., polyethylene), homopolymers and copolymers of hydrophobic aminoacids and derivatives of aminoacids (e.g., alanine, valine, isoleucine, leucine, norleucine, phenylalanine, tyrosine, tryptophan, threonine, proline, cistein, methionone, serine, glutamine, aparagine), homopolymers and copolymers of nucleic acid and derivatives thereof.

Preferred polyanionic carrier include those such as polymethacrylic acid and its salts, polyacrylic acid and its salts, copolymers of methacrylic acid and its salts, copolymers of acrylic acid and its salts, heparin, polyphosphate, homopolymers and copolymers of anionic aminoacids (eg. glutamic acid, aspartic acid), polymalic acid, polylactic acid, polynucleotides, carboxylated dextran, and the like.

Preferred polycationic carrier include polylysine, polyasparagine, homopolymers and copolymers of cationic aminoacids (e.g., lysine, arginine, histidine), alkanolamine esters of polymethacrylic acid (e.g., poly-(dimethylamrnonioethyl methacrylate), polyamines (e.g., spermine, polyspermine, polyethyleneimine, polypropyleneimine, polybutileneimine, poolypentyleneirmine, polyhexyleneimine and copolymers thereof), copolymers of tertiary amines and secondary amines, partially or completely quaternized amines, polyvinyl pyridine and the quaternary ammonium salts of the polycation segments. These preferred polycation segments also include aliphatic, heterocyclic or aromatic ionenes (Rembaum et al., Polymer letters, 1968, 6; 159; Tsutsui, T., In Development in ionic polymers-2, Wilson A. D and Prosser, H. J. (eds.) Applied Science Publishers, London, new York, vol. 2, pp. 167-187, 1986).

Additionally, dendrimers, for example, polyaridoamines of various generations (Tomalia et al., Angew. Chem., Int. Ed. Engl. 1990, 29, 138) can be also used.

Particularly preferred are copolymers selected from the following polymer groups: (a) segmented copolymers having at least one hydrophilic nonionic polymer and at least one hydrophobic nonionic segment; (b) segmented copolymers having at least one cationic segment and at least one nonionic segment; (c) segmented copolymers having at least one anionic segment and at least one nonionic segment (d) segmented copolymers having at least one polypeptide segment and at least one non-peptide segment; and (e) segmented copolymers having at least one polynucleotide segment and at least one segment which is not a nucleic acid.

In yet another preferred embodiment the invention provides a polypeptide capable of binding activated Mac-1 receptor, or derivative of the polypeptide, conjugated to a drug carrier system, such a carrier system being a polymer molecule, a block copolymer molecule, or a derivative of said polymer. The carrier system may also comprise a protein molecule. Preferred carrier systems are described elsewhere herein.

The preparation of the conjugates of the polypeptide or derivative to the therapeutic agent, or to the carrier system is effected by means of one of the known organic chemical methods for chemical ligation. The structural link between the polypeptide or derivative and the macromolecule, as well as the chemical method by which they are joined, should be chosen so that the binding ability of the polypeptide and the biological activity of the ligand, when joined in the conjugate, are minimally compromised. As will be appreciated by those skilled in the art, there are a number of suitable chemical conjugation methods. The selection of the appropriate conjugation method can be rationalized by the inspection of the chemical groups present in the conjugated molecules, as well as evaluation of possible modification of these molecules to introduce some new chemical groups into them. Numerous chemical groups can subject conjugation reactions. The following groups are mentioned here as examples: hydroxyl group (—OH), primary and secondary amino group (—NH₂ and —NH—), carboxylic group—(COOH), sulfhydryl group (—SH), aromatic rings, sugar residues, aldehydes (—CHO), alphatic and aromatic halides, and others. Reactivity of these groups is well known in the art (Morrison and Boyd, Organic Chemistry, 6th Ed. (Prentice Hall, 1992), Jerry March, Advanced Organic Chemistry, 4th Ed. (Wiley 1992), which are herein incorporated by reference). A more extensive description of conjugation methods and techniques can be found in: G. T. Harmanson, Bioconjugate Techniques, Academic Press, Inc. 1995, and in: S. S. Wong, Chemistry of Protein Conjugation and Cross-Linking, CRC Press, Inc. 1991, which are herein incorporated by reference.

Hydroxyl group—OH is present in peptides and proteins in side chains of serine, threonine, and tyrosine residues, and in sugar residues in sacharides and glycoproteins. Hydroxyl group is also present in many chemical compounds, including therapeutic agents such as paclitaxel, and in polymeric compounds, such as polisacherides and poloxamers. Hydroxyl groups exhibit nucleophilic properties and subject substitution reaction, for example alkylation (etherification), and acylation (esterification). The following reactive chemicals are preferred to conjugate with hydroxyls: alkyl halides (R—Cl, R—Br), cyanogen bromide (BrCN), acyl anhydrides, acyl halides, aldehydes (—CHO), hydrazides (R—CO—NH—NH₂), and others. Particularly preferred are: acyl anhydrides ((R—CO)₂O), and 1,1′-Carbonyldiimidazole (see: Anderson, G. W. and Paul, R., (1958) J. Am. Chem. Soc., 80, 4423, which is herein incorporated by reference).

Amino group —NH₂ is present in peptides and proteins at their N-terminus, if these are not acylated, and in side chains of lysine residues. Amino group is also present in many chemical compounds, including therapeutic agents such as doxorubicin. Chemical and genetic methods allow for introduction of amino group into numerous other molecules, including peptides, proteins, small organic molecules and polymeric molecules. Amino group reveals nucleophile properties, and it subjects substitution reaction, for example alkylation, acylation, and condensation with aldehydes. The following reactive chemicals are preferred to conjugate with amines: alkyl halides (R—Cl, R—Br, R—I), aryl azides, acyl anhydrides, acyl halides, acyl esters, carboxylates activated with carbodiimides, aldehydes (—CHO), and others. Particularly preferred are: acyl anhydrides ((R—CO)₂O), acyl chlorides (R—CO—Cl), p-nitropheny esters (R—CO—O—C₆H₄—NO₂), N-hydroxysuccinimidyl esters (NHS esters, R—CO—O—N(CO—CH₂)₂), imidoesters (R—C(═NH)—O—CH₃), and carboxylic acids activated with carbodiimides (R—CO—OH+R′—N═C═N—R″).

Sulfhydryl group —SH is present in peptides and proteins containing cysteine residues. Sulfhydryl group is also present in many chemical compounds, and can be introduced into other compounds (see for example Carlsson, J., Drevin, H. and Axen, R. (1978) Biochem. J. 173, 723). Sulfhydryl group subjects elecrophilic substitution reaction, for example alkylation, and oxidation reaction. Preferred are the following reactive chemicals, useful to conjugate with —SH group: alkyl iodides, unsaturated acyls, and oxidizing agents. Particularly preferred are the following derivatives: iodoacetamides R—CO—CH₂—I, maleimides (R—N(CO—CH)₂), vinylsulfones (R—SO₂—CH═.CH₂, Masri M. S. (1988). J. Protein Chem. 7, 49-54, which is herein incorporated by reference), didthiopyridyls (R—S—S-2-pyridyl).

Carboxyl group —COOH is present in peptides and proteins at their C-terminus (if not amidated), and in side chains of aspartic acid and glutamic acid residues. Carboxyl group is also present in many chemical compounds, including therapeutic agents such as methotrexate. Chemical and genetic methods allow for introduction of carboxyl group into numerous other molecules, including peptides, proteins, small organic molecules and polymeric molecules. Carboxyl group is able to acylate nucleophilic groups, such as amines and hydroxyls. Carboxyl group requires activation prior to conjugation. The preferred methods of activation are: reaction with organic or inorganic acid halides (for example pivaloyl chloride, ethyl chloroformate, thionyl chloride, PCl₅), reaction with carbodiimides (R—CO—OH+R′—N═C═N—R″, for example EDC, DCC), reaction with benzotriazolyl uronium or phosphonium salts (TBTU, BOP, PyBOP).

In preferred embodiment the conjugation of the polypeptide or derivative capable of binding activated Mac-1 receptor to other molecules, either a therapeutic agent or a drug carrier molecule, is achieved with the support of cross-linking reagent. Particularly preferred are heterobifunctional cross-linking reagents. Variety of cross-linking regents is known to those skilled in the art (see, for example, S. S. Wong, Chemistry of Protein Conjugation and Cross-Linking, CRC Press, Inc. 1991. which are herein incorporated by reference).

Heterobifunctional reagents are particularly useful for linking two molecule, one of them having amino group, and the other having sulfhydryl group. In a preferred embodiment the polypeptide or derivative capable of binding activated Mac-1 has a sulfhydryl group, and therefore is available for conjugation with variety of compounds bearing amino group. The following heterobifunctonal cross-linking reagents, for example, conjugate amino to sulfhydryl compounds: GMBS (N—[gamm.-Maleimidobutyryloxy]succinimide ester, Fujiwara, K., et al. (1988); J. Immunol. Meth. 112, 77-83)), SPDP(N-Succinimidyl 3-[2-pyridyldithio]propionate, Carlsson, J., et al. (1978). Biochem J. 173, 723-737), SIA (N-Succinimidyl iodoacetate, Thorpe, P. E., et al. (1984). Eur. J. Biochem 140, 63-71.), SVSB (N-Succinimidyl-[4-vinylsulfonyl]benzoate).

Particularly preferred heterobifunctional linkers have polyoxyethylene chain between the two reactive groups. Conjugation with such linkers yields products having hydrophilic junction between the two conjugated molecules, therefore it increases the solubility of the product in aqueous media. The following linkers with polyoxyethylene are mentioned here as examples: N-Maleimido-polyoxyethylene-succinimide ester (Sharewater Polymers, Cat. No. 2D2Z0F02), vinylsulfone-polyoxyethylene-succinimide ester (Shearewater Polymers, Inc. Al, Cat. No. 2Z5B0F02).

Methods of diagnosis are also included in the present invention, allowing detection of activated leukocytes by binding with a polypeptide or derivative as described herein. Accordingly, the invention further provides a method for detecting the presence, absence or level of an activated Mac-1 in a subject or a test article, the method including exposing the subject, or a biological sample of the subject or the test article, to a molecule, polypeptide or derivative thereof as described herein, and detecting binding of the molecule, polypeptide or derivative thereof to activated Mac-1.

The diagnostic methods can be used to diagnose and identify sites of potentially pathological Mac-1 activation (such as that occurring in inflammation or sepsis) in a subject. In light of this, the present invention further provides a method of diagnosis or prognosis of a Mac-1 mediated condition, the method including a method for detecting the presence, absence or level of an activated Mac-1 in a subject as described herein. In one form of the method, the Mac-1 related condition is sepsis.

As an initial investigation of diagnostic potential, the scFv MAN-1 was tested and demonstrated to be a marker of sepsis. Particularly in the early stages, this life-threatening clinical condition is difficult to diagnose due to the lack of conclusive laboratory parameters and due to a wide variation in clinical appearance. The activation of monocytes/macrophages plays a pivotal role in the pathogenesis of sepsis and early diagnosis and consequently early treatment can indeed change the outcome for patients.

Applicant proposes that activation-specific anti-Mac-1 scFv can detect monocyte activation and thus can be used to diagnose sepsis. In a pilot study comparing patients with severe sepsis with a sex- and age-matched control group without sepsis, the activation status of Mac-1 was significantly enhanced in patients with sepsis.

Other diagnostic applications relate to monocyte activation, such as in Wegener's granulomatosis, where disease activity correlates with the extent of Mac-1 expression of monocytes.

Mac-1 expression has also been shown to correlate with the risk of restenosis after coronary angioplasty, and to correlate with procoagulant activity after angioplasty in patients with acute myocardial infarction and to reflect the therapeutic effects of anti-platelet agents on monocyte activation after coronary stent implantation.

Overall, in immune response related diseases and in inflammation in general, activation-specific, anti-Mac-1 scFvs are proposed to be useful diagnostic tools. In terms of detecting a bound antibody molecule in diagnostic methods, a paramagnetic label could be coupled to a single chain antibody targeted to activated platelets. Upon administration of the coupled antibody, the paramagnetic label would localize at the site of elevated Mac-1 activation that could then be visualised by a magnetic resonance technique. Alternatively, the antibody could be radiolabelled (with technetium for example), with the activated leukocytes being visualized using a gamma camera. Also the labelling of activated leukocytes using computer tomography and ultrasonic methods (e.g. targeting of micro bubbles) is contemplated to be useful with the described peptides and polypeptides, derivatives thereof and antibodies. Other methods of detecting binding to Mac-1 such as (but not limited to) flow cytometry, ultrasound, gamma scintigraphy and computer tomography (such as positron emission tomography), and near-infrared detection are contemplated in the context of the method.

The skilled artisan will understand that the probe used for diagnostic and prognostic methods may be labelled by any method known in the art. Depending on the functionalization of the particles, different strategies can be used for this purpose. One way is to build peptide bonds between carboxy-functionalized SPIOs and free amino groups of the single-chain antibody. The skilled person is familiar with a range of commercially available coupling agents and kits that may be used for this chemical crosslinking approach. Another way would be to use the histidine-tag of the antibody for conjugation with commercially available cobalt-functionalized 1 μm SPIO-beads, whereby the single-chain antibody/bead complex is maintained by the binding of histidine to cobalt. Briefly, with this approach single-chain antibodies and SPIO-beads are incubated at room temperature for 10 minutes, thereafter the suspension is separated by a magnet and washed several times. Appropriate controls are generated by conjugating an irrelevant single-chain antibody to SPIOs using the same protocol.

The skilled person will understand that any probe useful in an X-Ray imaging method could be incorporated as a label. As a non-limiting example of the method, a paramagnetic label could be coupled to a probe targeted to activated Mac-1. Upon administration of the probe, the paramagnetic label would localize at the site of the activated Mac-1 that could then be visualised by a magnetic resonance imaging technique.

Alternatively, the probe could be radiolabelled (for example with technetium-99m, rubidium-82, thallium 201, F-18, gallium-67, or indium-111), with the activated Mac-1 being visualized using a gamma camera. Also the labelling of activated Mac-1 using computer tomography and ultrasonic methods (e.g. targeting of micro bubbles) is contemplated to be useful with the described molecules, peptides or polypeptides.

In another embodiment of the present invention provides a method of treating a condition associated with Mac-1 activation in a patient in need of such therapy comprising administering to the patient an effective amount of a pharmaceutical composition comprising at least one polypeptide or derivative thereof as described herein, wherein the polypeptide or derivative thereof is capable of specific binding with the high affinity Mac-1 receptor-1. The polypeptide may be a scFv substantially as described herein, or a shorter peptide substantially as described herein.

Diseases related to Mac-1 activation include (but are not limited to) inflammatory diseases which include, (but are not limited to) Crohn's disease, collitis ulcerosa, multiple sclerosis, sarcoidosis, psoriasis, atherosclerosis and its clinical sequelae, scleroderma, intestinal adhesions, hypertrophic scars, rheumatoid arthritis, septicemia, autoimmune disease, acute coronary syndrome, HIV infection, reperfusion injuries, ischemia, neointimal thickening, infiltration of polymorpholeucocytes, autoimmune disease, and neovascularisation-mediated disease. For instance, neointimal thickening after arterial injury was significantly reduced by antibody blockade of Mac-1 and in Mac-1 knock-out mice. Rats treated with a blocking anti-Mac-1 antibody demonstrate reduced ischemic cell damage after transient cerebral artery occlusion and Mac-1 deficient mice are less susceptible to cerebral ischemia/reperfusion injuries. Sepsis-induced lung infiltration of polymorphonuclear leukocytes (PMNs) was significantly reduced in a Mac-1 knock-out mouse model as well as in a transgenic mouse model in which NIF (neutrophil inhibitory factor), a blocking ligand of Mac-1, is over expressed. In autoimmune diseases Mac-1 blockade may offer a novel therapeutic approach. For example, in mice, autoimmune bullous pemphigoid could be prevented by antibody blockade of Mac-1 and by Mac-1 knock-out. For angiostatin, which has been shown to inhibit neovascularization, an anti-adhesive/anti-inflammatory effect mediated by the blockade of Mac-1 has recently been demonstrated. In particular, in skin injury/inflammation this mechanism may play an important role. Overall, an effective blockade of Mac-1 may allow targeting of chronic inflammatory processes in different pathologic settings.

While scFv molecules are proposed to be useful therapeutically, smaller antagonists will also have a role in treatment. It is proposed that small molecular weight inhibitors might be further developed to orally active compounds. As a first step towards creating a peptide mimetic, the paratopes of the single-chain antibodies were determined by mutational analysis. In MAN-1, two amino acids were identified (tryptophan and glycine), within the CDR3 region of the heavy chain that have a role in activation-specific binding. The fact that the same amino acids are also found in MAS-2 (Mac-1 activation-specific scFv obtained from the synthetic phage display library), one of the two clones of the synthetic single-chain library, underlines their role. In scFv clone MAS-1 the exchange of a centrally localized leucine by alanine reduces the binding to background level. In all three scFv clones the main paratope-forming amino acids are centrally located in the HCDR3 and are hydrophobic, providing structural features useful for the design of small molecular weight inhibitors. As a second step, HCDR3-derived peptides were produced and tested. Indeed, peptides derived from the HCDR3 of MAS-1 and MAS-2 displayed highly activation-specific inhibition of Mac-1. The inhibitory potency of these peptides were similar to HCDR3-derived peptides, which were developed as lead compounds for the therapeutic blockade of anti-p185HER2/neu for patients with breast cancer.⁴⁵ Notably, to the best of the Applicant's knowledge, to date all potent small molecule antagonists to I-domain-containing integrins are allosteric inhibitors, whereas the newly described HCDR3-derived peptides are based on a competitive inhibitory mechanism. Overall, it has been possible to define peptide sequences, which may serve as a basis for the development of orally active, activation-specific Mac-1 blockers.

The compositions and methods of the present invention can be used to treat patients with inflammatory bowel diseases such as Crohn's disease and ulcerative colitis. Both Crohn's disease and ulcerative colitis are characterized by chronic inflammation and angiogenesis at various sites in the gastrointestinal tract. Crohn's disease is characterized by chronic granulomatous inflammation throughout the gastrointestinal tract consisting of new capillary sprouts surrounded by a cylinder of inflammatory cells. Inhibition of angiogenesis by the compositions and methods of the present invention inhibits the formation of the sprouts and prevents the formation of granulomas.

Crohn's disease occurs as a chronic transmural inflammatory disease that most commonly affects the distal ileum and colon but may also occur in any part of the gastrointestinal tract from the mouth to the anus and perianal area. Patients with Crohn's disease generally have chronic diarrhea associated with abdominal pain, fever, anorexia, weight loss and abdominal swelling. Ulcerative colitis is also a chronic, nonspecific, inflammatory and ulcerative disease arising in the colonic mucosa and is characterized by the presence of bloody diarrhea.

The inflammatory bowel diseases also show extraintestinal manifestations such as skin lesions. Such lesions are characterized by inflammation and angiogenesis and can occur at many sites other than the gastrointestinal tract. The compositions and methods of the present invention are also capable of treating these lesions by preventing the angiogenesis, thus reducing the influx of inflammatory cells and the lesion formation.

Sarcoidosis is another chronic inflammatory disease that is characterized as a multisystem granulomatous disorder. The granulomas of this disease may form anywhere in the body and thus the symptoms depend on the site of the granulomas and whether the disease active. The granulomas are created by the angiogenic capillary sprouts providing a constant supply of inflammatory cells.

The compositions and methods of the present invention can also treat the chronic inflammatory conditions associated with psoriasis. Psoriasis, a skin disease, is another chronic and recurrent disease that is characterized by papules and plaques of various sizes. Prevention of the formation of the new blood vessels necessary to maintain the characteristic lesions leads to relief from the symptoms.

Another aspect of the invention provides for the use of a polypeptide or derivative thereof according to any one of claims 1 to 9 in the manufacture of a medicament for the treatment or prevention of an inflammatory disease. Preferably the conditionis selected from the group consisting Crohn's disease, collitis ulcerosa, multiple sclerosis, sarcoidosis, psoriasis, atherosclerosis and its clinical sequelae, scleroderma, intestinal adhesions, hypertrophic scars, rheumatoid arthritis, septicemia, autoimmune disease, acute coronary syndrome, HIV infection, reperfusion injuries, ischemia, neointimal thickening, infiltration of polymorpholeucocytes, autoimmune disease, and neovascularisation-mediated diseases.

In a further aspect the present invention provides a method for identifying a molecule capable of binding to activated Mac-1, the method including the steps of providing a library of candidate molecules, providing a first cell type exhibiting either activated Mac-1 or non-activated Mac-1, providing a second cell type exhibiting either activated Mac-1 or non-activated Mac-1, exposing the library of candidate molecules to the first cell type exhibiting non-activated Mac-1 and removing bound molecules to leave a first pool of molecules, exposing the first pool of molecules to the first cell type exhibiting activated Mac-1 and removing unbound molecules to leave a second pool of molecules, exposing the second pool of molecules to the second cell type exhibiting non-activated Mac-1 and removing unbound molecules to leave a third pool of molecules, exposing the third pool of molecules to the second cell type exhibiting activated Mac-1 and removing the unbound molecules to leave a fourth pool of molecules.

Preferably, the first cell type is a human leukocyte, and more preferably a monocyte. The second cell type may be a non-human cell type such as a Chinese Hamster Ovary (CHO) cell that has been engineered to express human Mac-1. Without wishing to be limited by theory, it is thought that using the differential panning strategy described above where a background of cell surface proteins provided by two different cell types, results in better selection of molecules capable of selectively binding activated Mac-1. The method may be used for the selection of scFv molecules, and particularly to select for conformation-specific antibodies. In contrast to classical panning strategies that are based on immobilized target molecules, a cell-based system was used allowing the display of complex, function-specific, transmembranous molecules consisting of multiple subunits.

Advantageously, the library is a phage library and phages in any of the pools created during execution of the method are amplified before the next step in the method. Phage display technology allows a subtractive approach, with depletion of phages that either bind non-specifically or that bind to Mac-1 in its non-activated state and selection of phages that bind to the activated Mac-1. To reduce unspecific background, a cell type was used that is distinct from human monocytes, but expresses Mac-1 either in a non-activated or an activated state. For this purpose, CHO cell lines were used that were either transfected with the native Mac-1 (for depletion) or with a mutated and thereby activated Mac-1 (for selection). The latter cell line has been developed in analogy to a cell line model based on a GFFKR-deletion in the integrin α-subunit that has been frequently used as a model for the activated GPIIb/IIIa (α_{IIb}β₃). This Mac-1-expressing CHO cells bearing a deletion of the GFFKR-region of the α_{M}-subunit demonstrate increased affinity to soluble ligands. The strategy to combine depletion and selection steps as well as the use of different cell backgrounds provides a unique specificity for a target molecule in defined conformational states, which can be used to target a wide variety of cell membrane proteins as well as protein complexes.

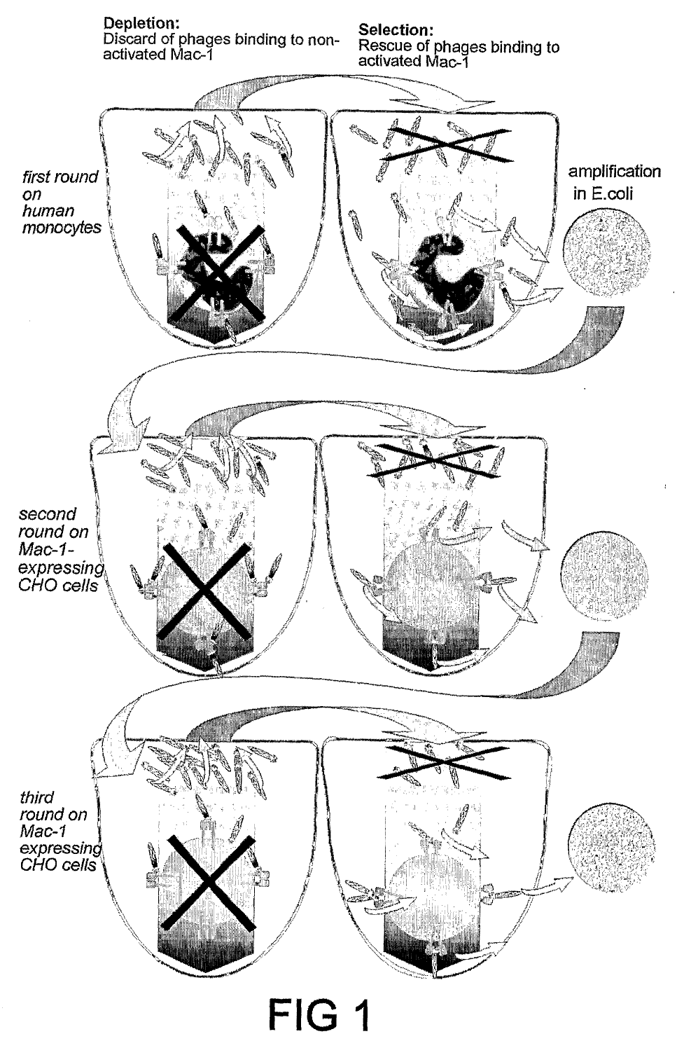

The panning method may be repeated any number of times, however, in a highly preferred form of the method three rounds of panning are implemented. A first round of panning is performed using monocytes, a second round uses CHO cells, and a third round uses CHO cells. A preferred method is described graphically in FIG. 1.

In yet a further aspect the present invention provides a molecule, peptide or polypeptide or derivative thereof capable of selectively binding to Mac-1, identified by a method as described above.

The invention will be now more fully described by reference to the following non-limiting Examples.

### Example 1

### Materials and Methods

The following materials and methods were used in Examples 2 et seq.

### Construction of Phage Libraries

A large natural phage display library of human scFv antibody fragments (natural library) was prepared in principle as described previously (Schwarz et al., 2004, Faseb J 18:1704-1706). Briefly V_{H} and V_{L} genes from cDNA from peripheral blood lymphocytes (PBL) of five healthy human donors and from spleen material of six additional donors were introduced in the phage surface display phagemid pEXHAM1. The resulting total complexity was about 1.8×10⁹ single clones (7.9×10⁸ clones PBL derived and 9.6×10⁸ spleen derived).

In addition a synthetic scFv library with mutated V_{H} chains was generated using two scFvs isolated from a large human scFv library as master frameworks. The V_{H} CDR3s of both master frameworks were replaced by synthetic DNA oligonucleotides containing the sequence TGT GCG ARA (NNK)₄₋₇ TTT GAS TAC encoding CDR3 loops of seven to 10 amino acids of the partly randomized amino acid sequence C A K/R X₄₋₇ F E/D Y. Oligos were cloned separately in pEXHAM1 generating libraries of 6.1×10⁸ single clones.

### Monocyte Isolation and Cells

Blood was collected by venipuncture with a 21-gauge butterfly needle from healthy volunteers taking no medications and was anticoagulated with citric acid. Isolated monocytes were prepared over Ficoll (Biochrom) gradients and separated from lymphocytes by adherence to plastic culture flasks placed in an incubator for 2 hours at 37° C. Monocytes were maintained in RPMI medium 1640 supplemented with 10% fetal calf serum (FCS), 100 units/ml of penicillin, 100 μl/ml of streptomycin, and 2 mM L-glutamine (all purchased from BioWhittaker).

Two Chinese hamster ovary (CHO) cell lines were generated expressing recombinant Mac-1 either as wild type (Mac-1 WT) or as a mutant (Mac-1 Del) with a GFFKR deletion of the α-subunit (CD11b). To introduce the deletion of the GFFKR-motif into the CD11b (α_{M}) cDNA, PCR was performed using the sense primer 5′-CCG CGC TGT ACA AGC TCC MT ACA AGG ACA TGA TGA GTG-3′ that excludes the nucleotides encoding for the amino acids GFFKR and introduces a BsrGI restriction site and the anti-sense primer 5′-TGC AAA AGC CTA GGC CTC CM-3′ that includes an AvrII restriction site. The DNA of wild type α_{M} served as a template in this reaction. CD11b (α_{M}) wild type and the GFFKR deletion were cloned into the expression vector pcDNA3, CD18 (β₂) was cloned into pZeoSV. CHO cells were transfected using Superfect™ transfection reagent (Qiagen) and clones were selected for resistance against 700 μg/ml G418 (Geneticine) and 250 μg/ml Zeocin® (both Invitrogen) and by the flow cytometric detection of CD11b and CD18 epitopes. Clones used in further experiments were examined by RT-PCR and immunoprecipitation to prove the correct surface expression of Mac-1 wild type (Mac-1 WT) and deleted Mac-1 (Mac-1 Del). To assure constant experimental conditions, the expression level of the transfected Mac-1 receptors on the CHO cells surface was monitored in parallel to each adhesion experiment in flow cytometry by anti-CD11b and anti-CD18 monoclonal antibodies (mAb).

CHO cells were maintained in Dulbecco's modified Eagle's medium (DMEM, BioWhittaker), 10% fetal calf serum (FCS), 2 mM L-glutamine, 100 U/ml penicillin, 100 μg/ml streptomycin, and 1% MEM nonessential amino acids (BioWhittaker). ICAM-1-expressing CHO cells were obtained from A. Duperray (Grenoble, France).

### Panning

All panning procedures were performed separately with the synthetic library and the natural library, respectively. The basic panning technique used herein is substantially as disclosed in Schwarz M et al Faseb J. 2004; 18:1704-1706. The first round of panning was performed on human monocytes: Initially, phages 1000 fold over the complexity of the starting libraries were added to 106 monocytes in Tyrode's buffer (150 mM NaCl, 2.5 mM KCl, 12 mM NaHCO₃, 2 mM MgCl₂, 2 mM CaCl₂, 1 mg/ml bovine serum albumin (BSA), 1 mg/ml dextrose; pH 7.4) and incubated two hours at room temperature. Then, monocytes were sedimented by centrifugation (20 min, 1000 g) and the supernatant was transferred to a fresh Falcon tube and incubated with fresh, washed monocytes, which were stimulated with 100 ng/ml PMA. After one hour incubation at room temperature the monocytes were washed twice in modified HEPES buffer (10 mM HEPES, 12 mM NaHCO₃, 138 mM NaCl, 2.9 mM KCl, 2 mM MgCl₂, 2 mM CaCl₂, 1 g/1 glucose, g/l BSA, pH=6.5). Bound phages were eluted by incubation with 0.1 M gylcine (pH=2.2) for 15 min, followed by neutralization with 1/10 volume of 2M Tris HCl (pH=8). The rescued phages were used for infection of log-phase XL-1-blue bacteria, which were plated on 14 cm agar plates containing 50 mM glucose, 100 μg/ml ampicillin and 20 μg/ml tetracycline. Resuspension, infection with M13 KO7 helper-phages, and polyethylene glycol (PEG)-precipitation were performed as described in Schwartz et al, ibid. Then, at least two rounds on Mac-1-expressing CHO cells were performed according to the protocol described above with the following modifications: For the depletion step, the phages from the previous round (1000× over the output number) were incubated with 2×10⁷ wild type Mac-1-expressing CHO cells in modified Tyrode's buffer and for selection GFFKR-deleted, “activated” Mac-1-expressing CHO cells (24) were used and further treated as described above.

Alternatively, heparinised blood was stimulated for 15 min at 37° C. with or without 100 ng/ml PMA and lysed with Lysing-Solution® (Becton Dickinson) following the manufacturers protocol. Then, the purified scFvs were added at various concentrations and incubated for 10 min. For the detection of scFv binding, a monoclonal Alexa Fluor 488 anti-His-tag antibody (Qiagen) was added and incubated for 10 min at room temperature. An anti-CD14-PE (Immunotech) double staining was performed to gate monocytes.

For the sepsis study (see Example 5) peripheral blood from 18 patients was used. The patients were diagnosed with severe sepsis as defined in a consensus document (Levy et al, 2001, SCCM/ESICM/ACCP/ATS/SIS International Sepsis Definitions Conference. Crit. Care Med 2003; 31:1250-1256) and from an age- and sex-matched control group without inflammation. Patients between 40 and 78 years old were recruited from the intensive care unit at the University of Freiburg, Germany. For statistical evaluation, the Mann-Whitney U test was applied (Prism v4.0, Graphpad Software).

Mac-1-expressing CHO cells were adjusted to 5×10⁶/ml and incubated with a PE-labeled anti-CD11b (2LPM19c, Dako), a FITC-labeled anti-CD18 (clone 7E4, Beckman), and MAN-1 (each 10 μg/ml) for 15 min at room temperature. MAN-1 binding was detected with the secondary anti-His-tag antibody as described above. Matched isotypes (Beckman) served as negative controls. CHO cells as well as monocytes were fixed with Cellfix® and analyzed in a FACS-Calibur® (all Becton Dickinson).

### High-Level Expression and Purification

Selected single-chain antibody clones were expressed and prepared as previously described. (Schwarz et al, Faseb J. 2004; 18:1704-1706). Briefly, the phagemid DNA was cloned, in the expression vector pHOG-21 using the restriction enzymes NcoI and NotI and transformed into TG-1 E. coli. These bacteria were grown at 37° C. to an optical density of 0.8 in LB-medium containing glucose (50 mM). Then, bacteria were transferred to LB-medium containing 0.4 M sucrose and incubated for 16 h at 200 rpm and 23° C. Finally, the bacteria were transferred to an ice-cold hyperosmotic shock solution (20% sucrose, EDTA, Tris) and incubated 1 hour on ice. Then, scFv was purified from other periplasmatic proteins by metal affinity chromatography using Ni-NTA-Agarose (Quiagen). Production and purification were monitored by SDS-PAGE and Western blotting.

### Flow Cytometry

Blood was diluted 1/50 in Tyrode's buffer, if needed activated by addition of 20 μM ADP, and then incubated for 20 min with various concentrations of purified scFv or 10 μl periplasmic product. Then, the suspension was incubated a second time for 20 min at room temperature with a monoclonal, FITC-labeled anti-His(6)-tag-antibody (Dianova), for detection of scFv binding or with a polyclonal FITC-labeled chicken-anti-fibrinogen antibody (WAK-Chemie, Bad Soden, Germany). After fixation with Cellfix (Becton Dickinson) samples were measured in a FACS-Calibur flow cytometer (Becton Dickinson).

### Fingerprinting, Sequencing and Screening

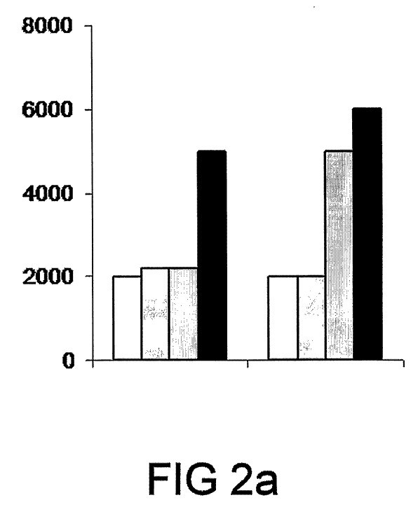

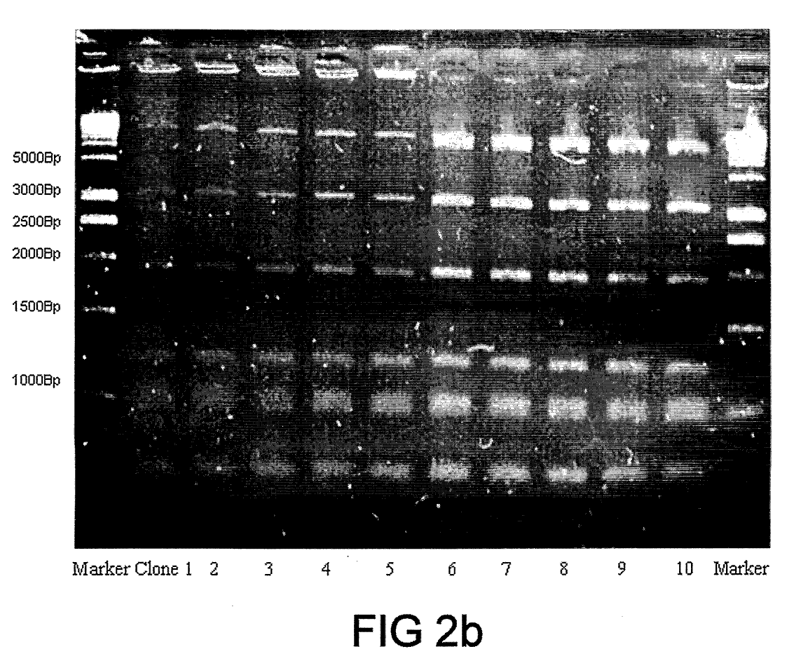

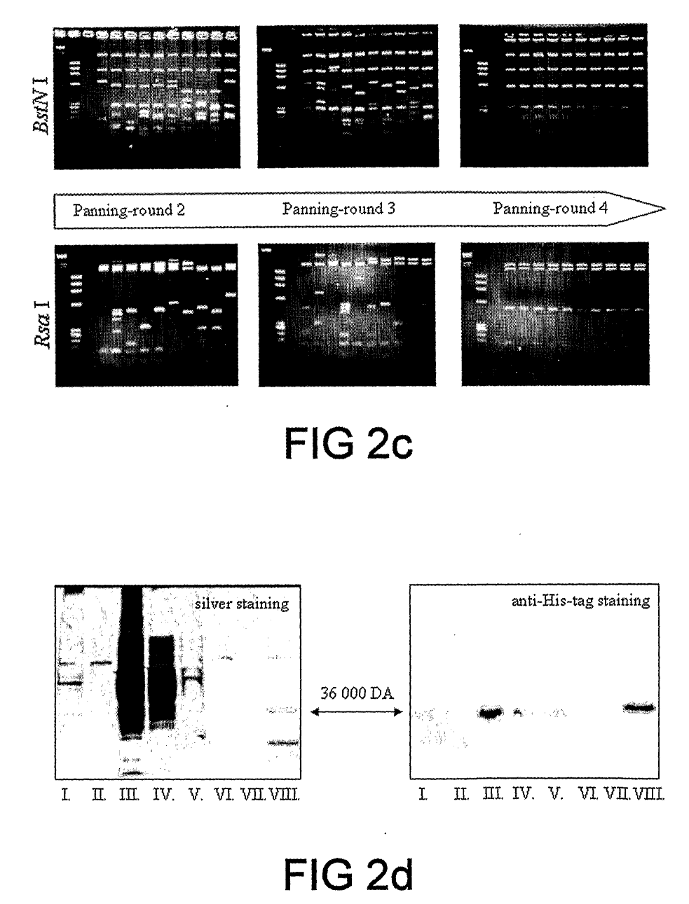

Phagemid-DNA of randomly picked natural clones was purified and digested with the BstNI restriction enzyme. Then, the scFvs of all individual clones were expressed in the periplasmic product of XL1-blue and small-scale periplasmic preparations were obtained. The periplasmic product was then dialyzed against PBS (14.000 MWCO, Spectrapor, Spectrum Laboratories) and tested in flow cytometry with human monocytes as described above. In parallel, the level of scFv expression was tested in Western blotting with an HRP-labeled anti-His(6)-antibody (Roche). Finally, all clones expressing a binding scFv were sequenced using an automated DNA sequencer. Alignment of the amino acid sequences was performed using the Clustal multiple alignment program (HUSAR-package, biocomputing group, German Research Cancer Institute, Heidelberg, Germany). Screening of clones by restriction analysis, sequencing, and purification is shown in FIGS. 2a to 2d. Considering FIG. 2d it will be seen that the bacterial suspension contains a low concentration of scFv (lane 1). After centrifugation, the scFv is not detectable in the supernatant (lane 11) but at a high concentration in the lysate of the bacterial pellet (lane II). The flow through of the Ni-NTA-agarose-column (shown in lane IV) shows a large amount of protein (silver staining), but no signal in the His-tag staining, indicating that the scFvs are bound to the Ni-NTA-agarose, whereas unspecific proteins flow through. The following washing steps (lane V and VI) demonstrate further wash out of decreasing amounts of proteins that include only a small portion of scFv. The comparison between the bacterial suspension (lane 1) and the final eluate (lane VIII) demonstrates the power of bacterial protein expression/isolation and the Ni-His-tag purification system.

### Peptide Synthesis.

Solid-phase peptide synthesis was performed with sequences derived from the HCDR3 regions of MAS-1 and MAS-2 or the described sequence within the I-domain of α_{M} on an Applied Biosystems 433A peptide synthesizer by Fmoc-strategy. For synthesis of cyclic peptides, a cystein residue was added at each end of the sequence. Peptides were purified by HPLC on a Vydac (Hesperia) C₁₈ reversed-phase preparatory column and characterized by analytical HPLC and MALDI-MS.

### I-Domain Peptide ELISA

The peptide sequence KFGDPLGYEDVIPEADR mimics the binding site for the P2-C sequence of Fibrinogen within the M I-Domain of Mac-1 (Yakubenko et al., 2001), was synthesized and conjugated to ovalbumin (PSL), diluted in coating buffer containing 1.6 g/l Na₂CO₃, 3 g/l NaHCO₃ at a pH of 9.6 to a concentration of 20 μg/ml. A 96 well plate (Nunc ImmunoPlate, MaxiSorp®) was coated with 200 μl of this solution over night at 4° C. Wells coated with ovalbumin not coupled to the I-domain peptide served as blank. Subsequently, the wells were washed with 5 rounds of pipetting with washing buffer containing PBS (Bio Whittaker Europe) with 0.05% Tween20 (Roth) and blocked with 300 μl/well of PBS with 1% BSA (SERVA) for 2 hours. After washing 3 times with washing buffer 100 μl/well of MAN-1 diluted in incubation buffer (PBS w. 2% BSA) to a concentration of 4 ng/100 μl were added and incubated overnight at 4° C. A scFv-antibody (clone MA2) that does not bind to Mac-1 was used as control (Schwarz et al., 2004; ibicl). Before staining and after each staining step the wells were washed 6 times with washing buffer by pipetting. 100 μl of anti-His(6)-tag antibody (Quiagen, Penta-His-antibody) were added and incubated for 2 hours. After another washing step 100-μl of an anti-mouse-antibody-HRP-conjugate (Pierce) were added. After 1 hour the wells were washed and 100-μl of TMB-Substrate (Pierce) were added. After additional 20 minutes incubation at 37° C., the reaction was terminated by the addition of 100 μl 2M H₂SO₄/well. Absorption was measured in an ELISA plate reader at 450 nm.

### Adhesion Assays

Adhesion assays were performed on fibrinogen, heparin and C3bi. 96 well plates (Nunc ImmunoPlate, MaxiSorp®)) were incubated with 100 μl of 100 U/ml Heparin in PBS (pH 7.4) or 5011l of 20 μg/ml fibrinogen-solution in PBS, overnight at 4° C. After two rounds of washing with PBS, fibrinogen-coated plates were blocked with 100 μl aliquots of 0.1% agarose, heparin-coated plates were blocked with 100i of 1% BSA in PBS, both for 1 hour at room temperature. Mac-1 Del cells, and as a negative control, untransfected CHO cells were resuspended in PBS at a concentration of 1 million/ml. Cells were preincubated with or without blocking antibodies for 10 minutes at room temperature. All antibodies were added at a concentration of 10 μg/ml. CD11b mAb Lpm19C (Dako Cytomation) served as positive control for maximum blocking ability and an unspecific scFv-antibody as negative-control.

100 000 cells per well were allowed to adhere on immobilized heparin or fibrinogen for 30 minutes at room temperature. Then, the non-adhering cells were washed off with two rounds of pipetting. The residual adherent cells were quantified with the following colorimetric assay: The cell-endogenous acid phosphatase activity was used by adding 100 μl of the following substrate/lyses solution to each well: 1% Triton X-100, 6 mg/ml p-nitrophenylphosphate (Sigma), in 50 mM sodium acetate buffer, pH 5. After 1 hour incubation at 37° C., the reaction was terminated by the addition of 50 μl of 1 M NaOH. The plate was read in an ELISA plate reader with a 405 nm filter.

Evaluation of cell binding to C3bi was performed as described previously by Shimaoka et al, Proc Natl Acad Sci USA 2002; 99:16737-16741, and Shimaoka et al Nat Struct Biol. 2000; 7:674-6781. Preserved binding of a neo-epitope-specific anti-C3bi antibody obtained from Quidel, suggests that C3bi is functionally intact after immobilization (data not shown).

C3bi-coated plates were blocked with 1% BSA. CHO cells expressing activated Mac-1 and, as a negative control, non-transfected CHO cells were preincubated with or without blocking antibodies (10 μg/ml) for 10 min at room temperature. Anti-CD11b mAb 2LPM19c served as positive control and an unspecific scFv as negative control. 100 000 cells per well were added and incubated for 30 min at 37° C. Non-adherent cells were washed off. Cell adhesion was quantified as described elsewhere (Ahrens et al Exp Cell Res. 2006; 312:925-937). To evaluate Mac-1-mediated adhesion to ICAM-1, a monolayer of ICAM-1-expressing CHO cells was used after blocking with 1% BSA. CHO cells expressing Mac-1 in the activated or non-activated state, which were partially preincubated with blocking antibodies, were allowed to adhere for 30 min at 37° C. After two washing-steps, adherent Mac-1 cells, which were still in the round, unspread state, were counted in 6 visual fields. Adhesion to a monolayer of non-ICAM-1-expressing CHO cells served as blank values.

### Analysis of Mac-1-Mediated Cell Adhesion Under Flow Conditions

Adhesion of recombinant Mac-1-expressing CHO cells or human monocytes to a fibrinogen matrix under shear stress was assessed using a modified parallel plate flow chamber assembly (GlycoTech) described by Lawrence et al Blood. 1987; 70:1284-1290. Fibrinogen (100 μg/ml) was immobilized on rectangular cover slips overnight at 4° C. at a concentration of 100 μg/ml. After 2 rounds of washing with PBS, the cover slips were blocked with 1% filter-sterilized BSA for 1 hour at room temperature. CHO cells and monocytes were adjusted to 1 million/ml. Monocytes were pre-incubated with PMA 100 ng/ml for 15 minutes. CHO cells were adjusted to 1 million/ml.

Cells and monocytes were pre-incubated with MAN-1 (20 μg/ml), with cyclic peptides either derived from MAS-1 or MAS-2 (10 or 100 μM), or with an activation-unspecific blocking anti-CD1b antibody (2LPM 19c, 10 μg/ml) (DAKO-Cytomation) or no addition for 15 minutes prior to perfusion. Afterwards, each differently pre-treated cell line was injected separately into a flow chamber and pre-adhesion was allowed for 7 minutes. Then, using a syringe pump (Harvard Apparatus Inc.) the cell suspensions were perfused through the parallel plate flow chamber for one minute at a shear rate of 0.5 dyne/cm², simulating venous flow, followed by one minute at 15 dyne/cm², simulating arterial flow. Monocytes were directly perfused into the chambers, without pre-adherence starting with 2 minutes of slow-perfusion at 0.02 ml/min and then at the same two shear-rates as the CHO cells for one minute each. Temperature was maintained at 37° C. Cell and monocyte adherence was visualised in real-time for up to 6 min by phase contrast microscopy (63×/oil objective) using a Zeiss Axiovert-200 epifluorescence microscope (Carl Zeiss) and images were captured in real time using a liquid-chilled CCD camera. Images were analysed offline using the commercial software package MetaMorph (version, 4.6.8, Universal Imaging Corp.). The adherent cells/visual field after 30 seconds of flow were counted and evaluated. In some experiments adherent cells were counted after 3 min of venous flow and 1 min after the application of arterial flow

### Alanine Substitution PCR of the Heavy Chain CDR3 Region of MAN-1

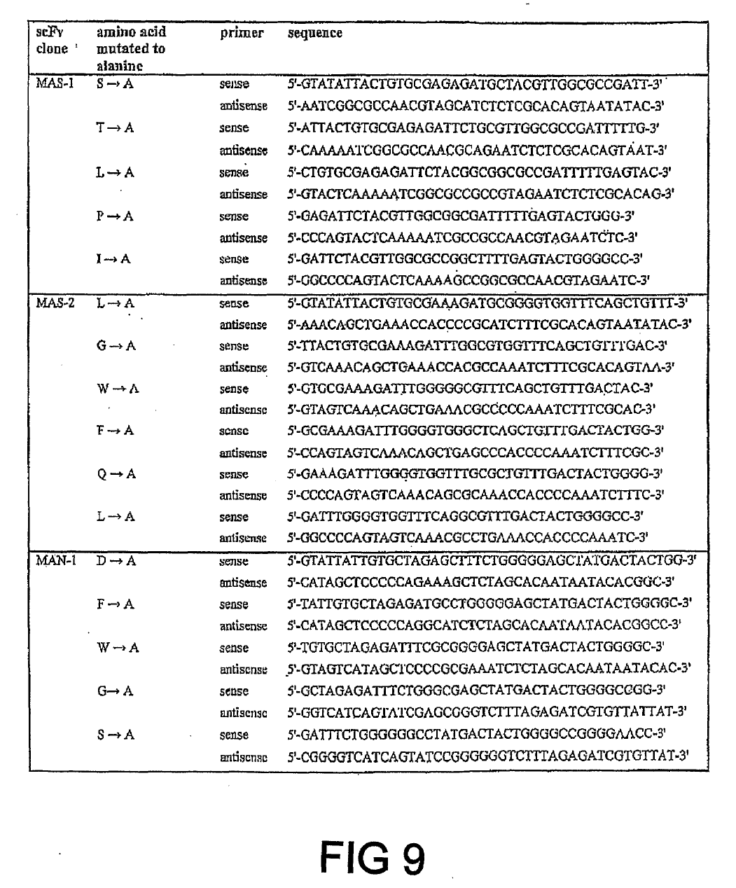

To evaluate the role of single amino acid residues for the binding of the scFv an alanine scan of the HCDR3 was performed. Using mutagenic primers (FIG. 9), each of the five amino-acids of the heavy separately replaced by alanine. PCR was performed with a sense primer binding at the beginning of the scFv-sequence and the antisense primer binding directly at the CDR3 of the heavy chain including the desired mutation and the PinAI restriction site at the 5′-end. The obtained PCR products were then cloned into the initial plasmid vector (pHOG) with NcoI and NotI. Mutagenesis of the amino acids included in the PinAI restrictions site was performed with a mutagenic primer including the desired mutation and amplification of the whole plasmid (Quickchange kit, Stratagene). The binding characteristic of these mutated scFvs was evaluated with flow cytometry as described above.

### Example 2

### Generation of Conformation-Dependent Antibodies by Phage Display

Starting material were two previously described single-chain antibody phage libraries. One so-called natural phage library was based on cDNA from peripheral blood lymphocytes of healthy human donors and from spleen material. Using PCR the variable regions of the antibodies' heavy and light chain were amplified. The second library, the so-called synthetic library, was generated by insertion of randomized synthetic nucleotide-sequences in the complementary-determining region 3 (CDR-3) of the variable region of the heavy chain (V_{H}) of two well-established scFvs (E4 and C5). Overall, library complexities of up to 10⁹ single clones were achieved.

To select phages directed against activation-specific epitopes of the large, complex (two non-covalently coupled subunits) cell surface molecule Mac-1, a novel panning strategy was developed to reduce non-specific binding and to select for activation-specific scFvs (see FIG. 1). A procedure was established including the following unique features: (1) To wash off unspecifically binding phages, a washing buffer with a relatively low pH (6.5) was used. (2) To present the receptor with a distinct background, panning was performed in series with Mac-1 expressed on two different cell types (monocytes and Mac-1 transfected CHO cells), which are composed of a completely different cell surface background. (3) To deplete all phages binding to non-activated receptors or to the cell background, the phage suspension was primarily incubated with non-activated Mac-1 on monocytes or CHO cells. After centrifugation, these cells including the phages that bound to these cells were discarded.

The natural and synthetic libraries demonstrated a very similar course of panning (FIG. 2a): In the first round of panning, the method started with 1.8×10¹², respectively 7.5×10¹¹ phages (corresponding to 1000× over the initial complexity of the libraries). With both libraries only about 2000 phages were selected. In the second round essentially there was no change and still only a low number of phages were selected. In the third round the synthetic library already demonstrated increased colony counts, whereas the natural library again contained only about 2000 clones. Finally, in the last round a significant increase of selected phages of up to 6000 clones was noted. The increase of clones after panning round 4 as shown in FIG. 2a, represents the amplification of a few very strongly binding clones, which bind to the activated receptor but not to the inactivated Mac-1 receptor and therefore were amplified through the course of panning leading to the increase of bound phages and thus leading to an increase of colonies after re-infection of E. coli.

The diversity of the natural clones was identified by the distinct patterns obtained by digestion with the restriction enzyme BstNI (FIG. 2b): About 20 randomly picked clones demonstrated only one restriction-pattern indicating the enrichment of essentially one single clone. For characterization of the synthetic clones 10 randomly picked clones were sequenced. This revealed two distinct clones, equally distributed (five each). Altogether, it was possible to enrich one natural (MAN-1: Mac-1 activation-specific scFv obtained from the natural library) and two synthetic clones (MAS1 & 2: Mac-1 activation-specific scFv obtained from the synthetic antibody library) by differential panning. The full length sequence of MAN-1 is depicted in FIG. 3a. Since in the synthetic library the HCDR-3 was randomized and two predefined frameworks were used otherwise, for MAS1 and MAS2 only the HCDR-3 sequences are given in FIG. 3b. The frameworks of MAS1 and MAS2 are derived from the previously published scFv E4.

### Example 3

### Alanine Scan of the HCDR-3 Region of Man-1

Since in most antibodies the CDR-3 region of the heavy chain (HCDR-3) is a main determinant of epitope recognition, the role of this region in the scFv MAN-1 that was derived from the natural single-chain antibody library was evaluated. By changing the individual amino acids of the HCDR-3 to alanine (alanine scan) it was possible to address the role of the region itself as well as the role of the individual amino acid for the activation-specific epitope recognition of MAN-1. Using PCR, alanine mutant clones of MAN-1 were created, expressed in TG-1 E. coli, purified and evaluated in flow cytometry. The replacement of the first two and the last amino acid of the HCDR-3 showed a slight reduction in the binding ability of MAN-1 scFv to activated Mac-1 (FIG. 4). The substitution of the two central amino acids resulted in a significant loss in the binding to Mac-1 (FIG. 4). This result emphasizes a role of the HCDR-3 and point to a role for the two central amino acids within the HCDR-3, at least for the epitope that is the target of MAN-1. The importance of the centrally located tryptophan (W) is underlined by its appearance in the clone MAS-2. In an alanine scan of this clone, the replacement of this amino acid led to a loss of binding of the clone. For MAS-1 the centrally located leucin (L) seems to be the essential amino acid within the HCDR-3. The mutations suggest that at least a component of the interaction of the scFv with Mac-1 is hydrophobic.

### Example 4

### Flow Cytometric Demonstration of Activation-Specific Binding to Mac-1

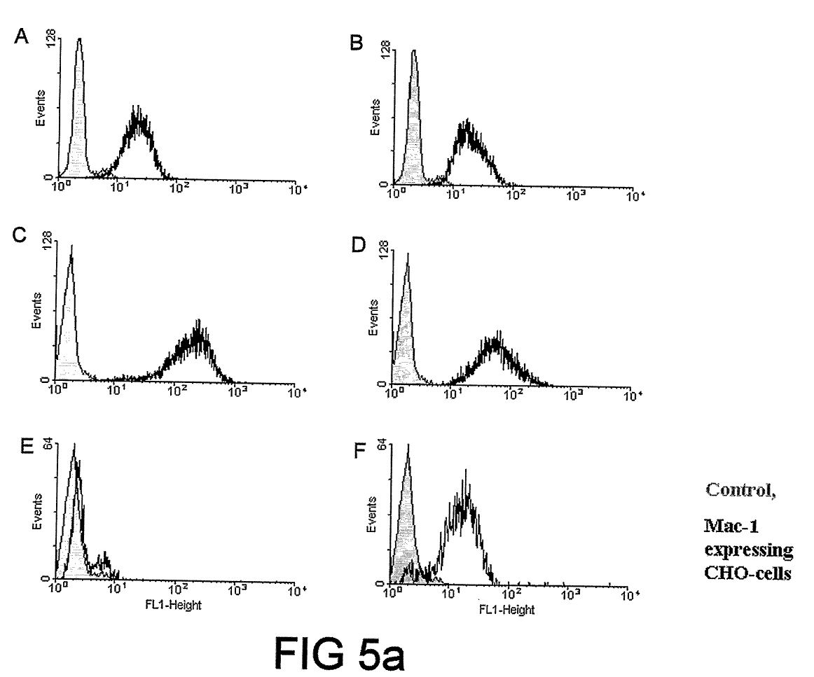

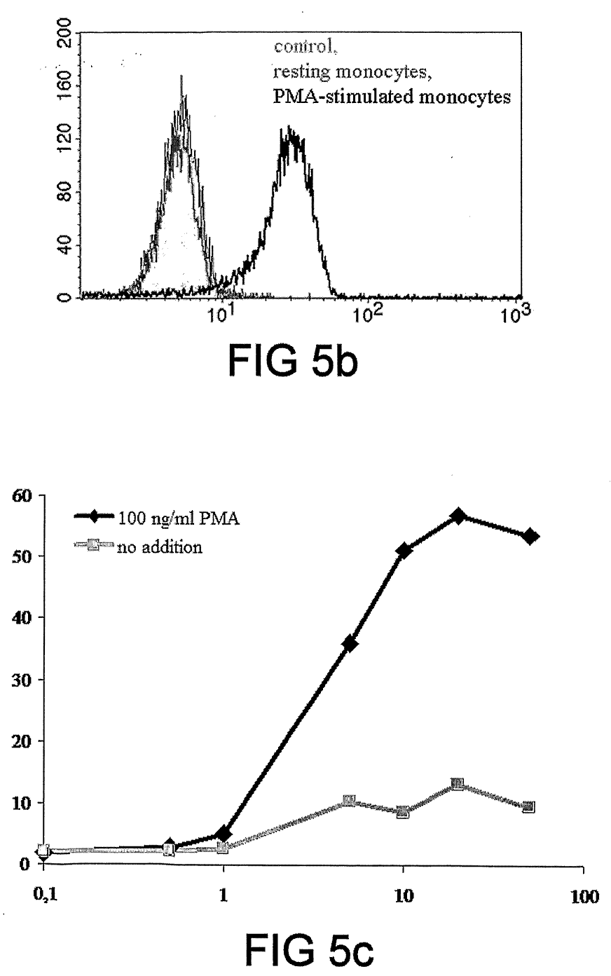

All individual clones were expressed in E. coli TGI and purified using IMAC (immobilized metal affinity chromatography). A highly purified protein with 36 kDa was obtained. The binding to Mac-1-expressing CHO cells was tested in flow cytometry. MAN-1 demonstrated no specific binding to CHO cells that express wild type Mac-1 (FIG. 5a). In contrast, MAN-1 strongly bound to the CHO cells expressing the GFFKR-deleted and thus activated receptor (FIG. 5a). Furthermore, monocytes obtained from healthy donors were used to prove the activation-specific binding of MAN-1 to Mac-1. MAN-1 demonstrated a strong binding to PMA-activated but not to non-activated monocytes (FIGS. 5b,c). Selective binding to the activated Mac-1 was maintained at concentrations beyond the saturation level (FIG. 5c). Similar binding properties proving activation-specific binding to Mac-1 were found with the clones MAS1 and MAS2 (data not shown).

### Evaluation of Potential MAN-1 Cross-Reactivity.

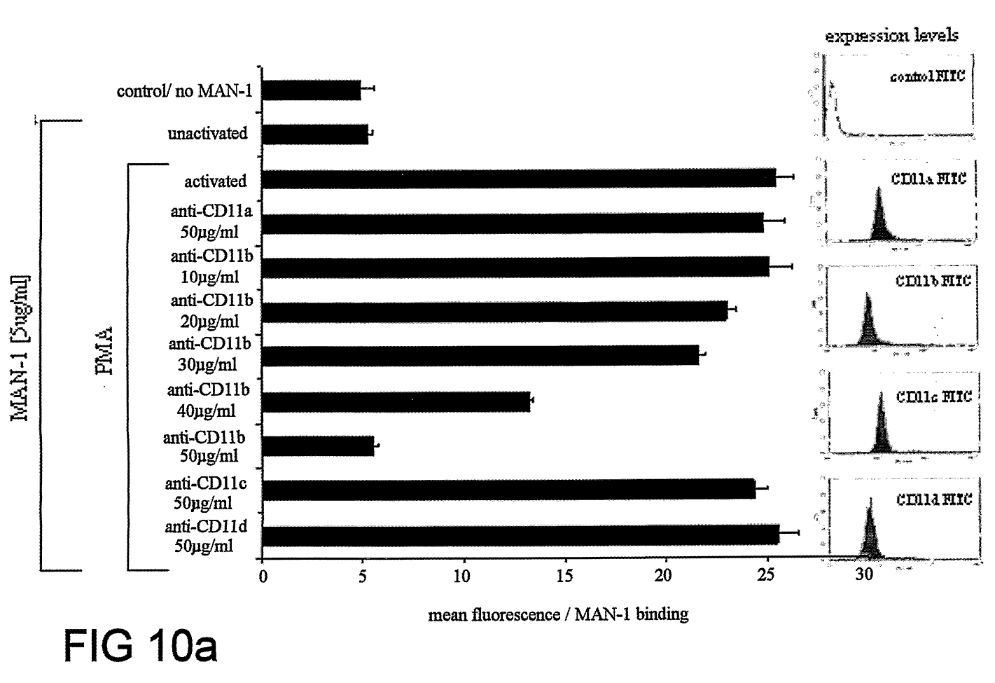

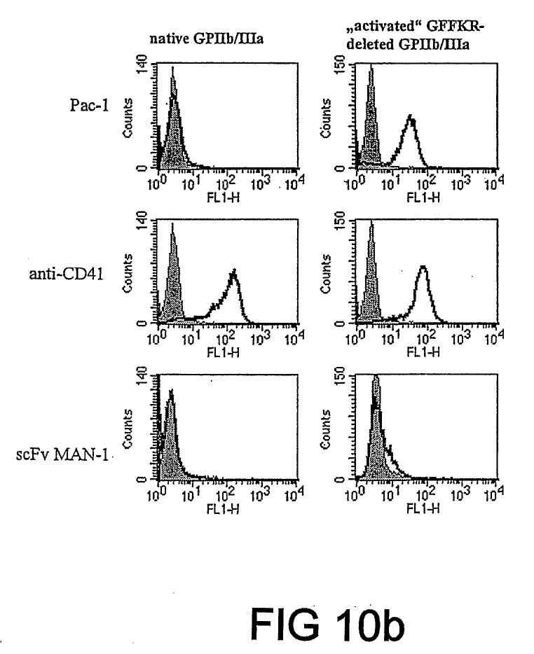

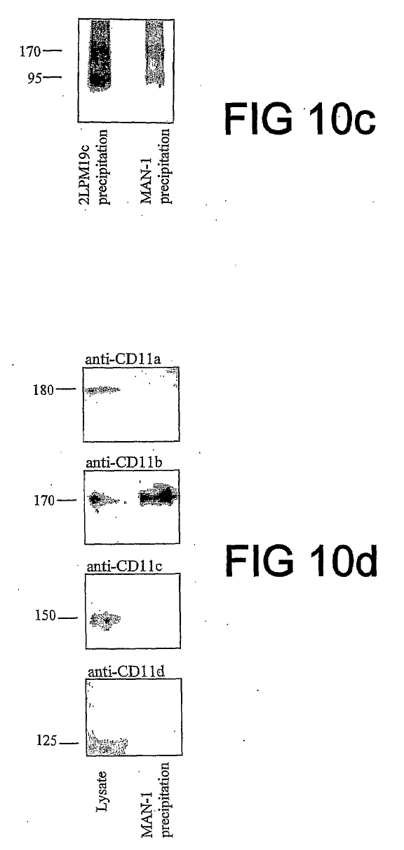

Since the group of β₂-integrins are structurally related and share common ligands, MAN-1 may bind to other 2-integrins besides Mac-1. In a flow cytometric assay MAN-1 binding to activated monocytes is not inhibited by antibodies described to block the β₂-integrins LFA-1 (α_{L}β₂, CD11a/CD18), p150,95 (α_{X}β₂, CD11c/CD18), or α_{D}β₂ (CD11d/CD18) as demonstrated in online FIG. 10. In contrast, MAN-1 binding is blocked by an anti-Mac-1 antibody in a concentration-dependent manner up to a full blockade, suggesting that MAN-1 binding is specific for the β₂-integrin Mac-1 (FIG. 10a). Potential cross-reactivity was further addressed by immunoprecipitation of Mac-1 from lysed, PMA-activated monocytes using MAN-1 as the precipitating antibody (FIGS. 10c, d). Two bands were precipitated that fit to the molecular weight of the α_{M} subunit with around 170 kDa and the β₂-subunit with around 95 kDa (FIG. 10d). This suggests that there is no significant binding of MAN-1 to α_X or α_D, which would otherwise result in precipitates at 145 kDa and 125 kDa, respectively. Since the molecular weight of α_L is described to be close to α_M (˜170 kDa), the immunoprecipitation experiment can not exclude binding of MAN-1 to LFA-1. For this reason and to further support the finding that there is no significant binding to α_{X} or α_{D}, Western blot analysis was performed on MAN-1 precipitates of lysed, PMA-activated monocytes using anti-CD11a, anti-CD11b, anti-CD11c and anti-CD11d antibodies. Whereas the monocyte lysate stained positive for all of the □2 integrins, the MAN-1 immunoprecipitate did not show an appropriate band for CD11a, CD11c and CD11d. In contrast, an anti-CD11b antibody was clearly positive with both the monocyte lysate as well as the MAN-1 precipitate (FIG. 10d). Thus, competition experiments of MAN-1 with blocking antibodies against all the members of the β₂-integrin family as well as immunoprecipitations and Western blots suggest a selective binding of MAN-1 to the β₂-integrin Mac-1.

Since one of the Mac-1 ligands, fibrinogen, also binds to the platelet integrin GPIIb/IIIa (α_{IIIb}β₃, CD41/CD61) potential cross-reactivity of MAN-1 with this integrin was also evaluated. For this purpose, recombinant CHO cell lines were used, expressing native, non-activated and GFFKR-deleted, activated GPIIb/IIIa as described elsewhere (O'Toole et al J. Cell Biol. 1994; 124:1047-1059; Peter et al, J Exp Med. 1995; 181:315-326; Tadokoro et al Science. 2003; 302:103-106). Whereas, the activation-specific monoclonal antibody Pac-1 (Tadokoro et al, ibid) binds to the activated GPIIb/IIIa, MAN-1 does not bind to GPIIb/IIIa, neither in the activated nor in the non-activated state (FIG. 10b).

### Example 5

### Localisation of Man-1 binding to the Mac-1 I-Domain

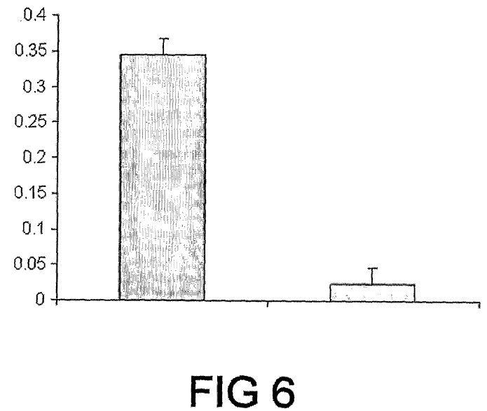

The I-domain is proposed to be the main activation-specific binding site of the Mac-1 receptor. Since it has been shown that the I-domain is exposed after the conformational change associated with Mac-1 activation and since the newly designed selection procedure described herein aiming to select for activation-specific scFvs, Applicants proposes that (and without wishing to be limited by theory) the main epitope for scFv MAN-1 is the I-domain. An I-domain peptide was immobilized and binding of MAN-1 was evaluated. The scFv MAN-1 demonstrated a specific binding to the I-domain peptide compared to a control scFv (FIG. 6). Further experiments have shown ScFv MAN-1 demonstrates a concentration dependant binding to the I-domain peptide compared to a control peptide, which was a scrambled (randomized) I-domain peptide sequence (FIG. 12). This localizes the binding region for scFv MAN-1 to the I-domain-region Lys²⁴⁵-Arg²⁶¹ of Mac-1.

### Example 6

### Cell Adhesion Assays

### Adhesion Assays Under Static Conditions

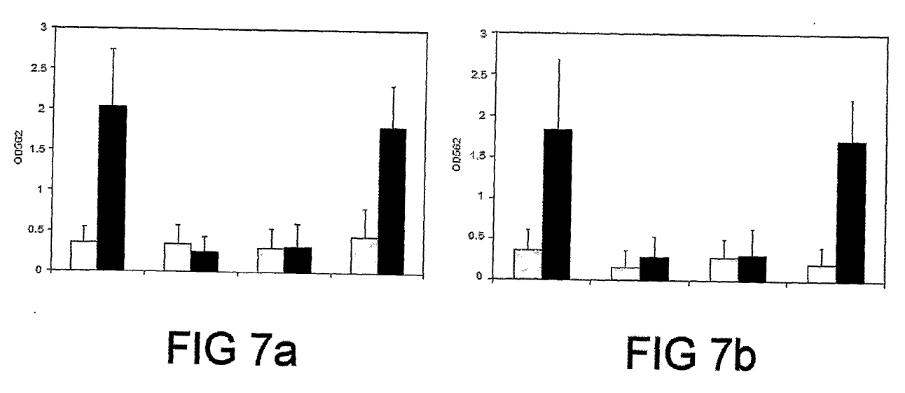

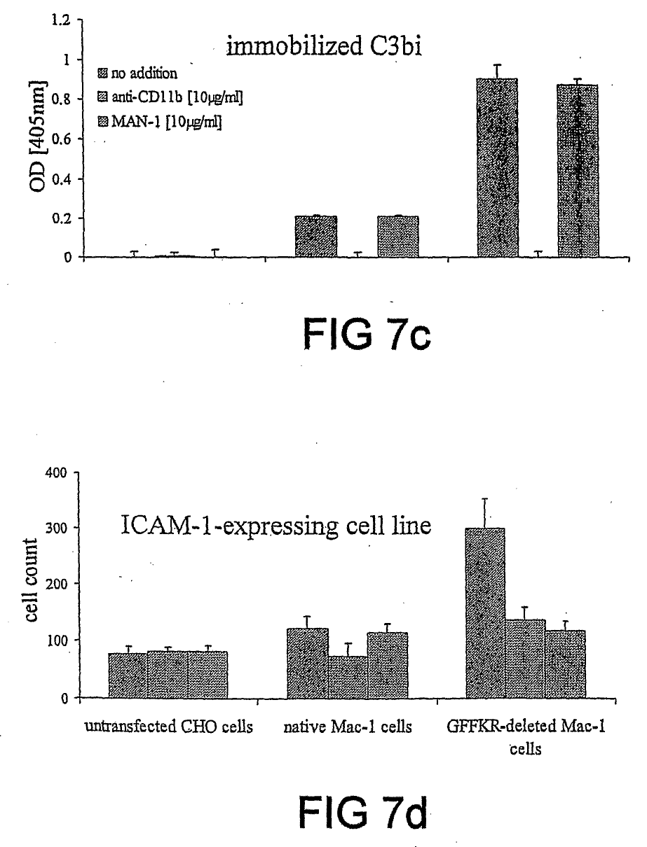

To evaluate the ability of the MAN-1 scFv to block ligand binding to Mac-1, adhesion assays on the Mac-1 ligands fibrinogen, heparin and C3bi were performed. The adhesion of CHO cells, which express activated Mac-1, to fibrinogen and heparin could be blocked by MAN-1 to the same extent as by an anti-CD11b antibody (FIG. 7). Thus, the scFv MAN-1 has ligand-blocking properties.

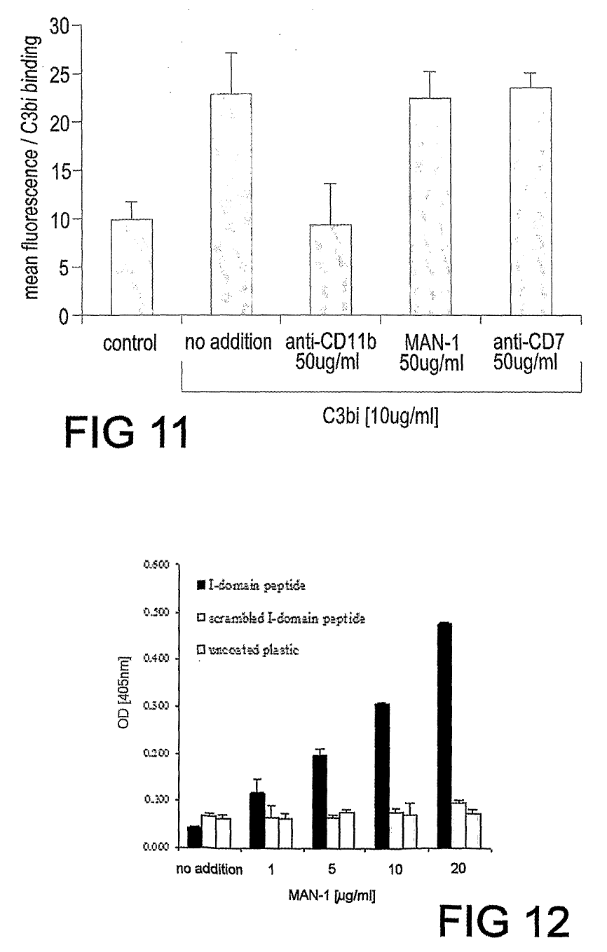

The binding of the activated Mac-1 to immobilized C3bi could only be blocked by the activation-unspecific anti-CD11b antibody, but not by MAN-1 (FIGS. 7c, d). Furthermore, flow cytometric measurements demonstrated that binding of soluble C3bi to PMA-activated monocytes is not inhibited by MAN-1, whereas an anti-CD11 b antibody was able to block C3bi binding to activated monocytes (FIG. 11).

In an ICAM-1 adhesion assay, MAN-1 was able to inhibit the binding of the activated Mac-1 transfected cell lines, but not the background binding of non-activated Mac-1 to an ICAM-1-expressing cell line, as opposed to the unspecific anti-CD11b antibody, which inhibited both conformations (FIGS. 7c, d). Thus, MAN-1 inhibits ICAM-1 binding to activated Mac-1.

### Adhesion Assay Under Flow Conditions

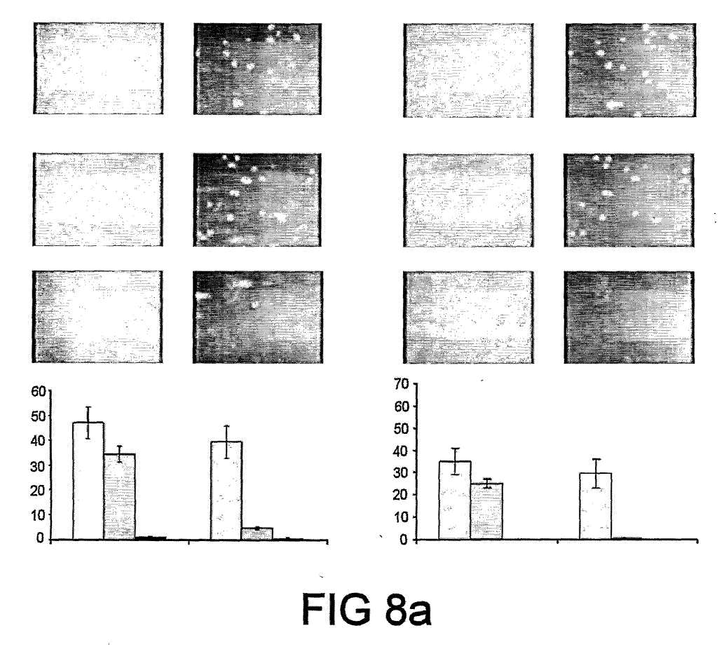

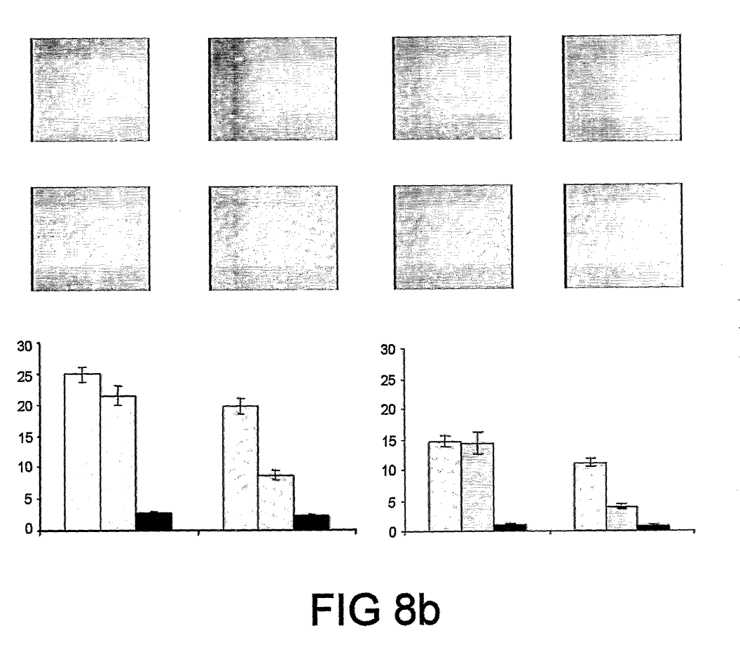

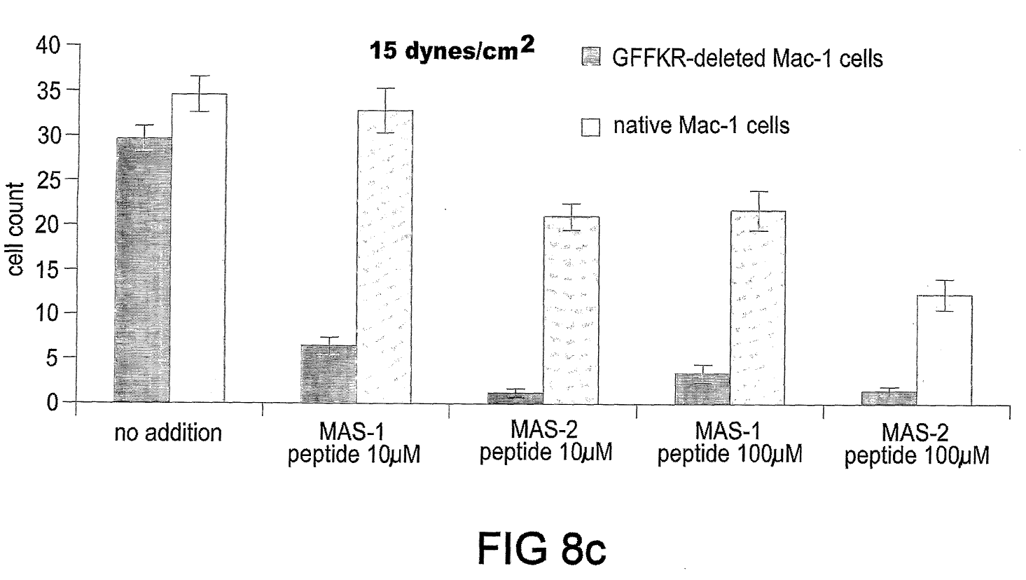

To further evaluate the potential therapeutic use of MAN-1, the activation-specific blocking effect of MAN-1 was investigated under flow conditions. CHO cells expressing native Mac-1 or GFFKR-deleted Mac-1 were tested for adhesion on immobilized fibrinogen under low as well as high flow rate. MAN-1 selectively blocked adhesion of CHO cells that express the GFFKR-deleted and thereby activated Mac-1 (FIG. 8a). To provide additional data on the activation-specific blockade of the Mac-1 integrin, flow chamber experiments were performed comparing adhesion on immobilized fibrinogen of activated and non-activated monocytes. Indeed, only adhesion of the PMA-activated monocytes was inhibited (FIG. 8b). The adhesion of monocytes to fibrinogen persisted under the arterial flow rate, which was applied at the end of the recording time. As expected, only adhesion of the PMA-activated monocytes was inhibited by MAN-1. Adhesion of non-activated monocytes was unaffected.

Whereas, adhesion of the non-activated monocytes was unaffected (FIG. 8b). Thus, as aimed for by the newly designed scFv selection procedure, the activation-specific binding of MAN-1 allows an activation-specific inhibition of the activated Mac-1 and of activated monocytes. This experiment was also performed with the CHO cells expressing native Mac-1 or GFFKR-deleted Mac-1 for adhesion on a fibrinogen-matrix as described previously²⁸. MAN-1 selectively blocked adhesion of CHO cells that express the GFFKR-deleted and thereby activated Mac-1 (FIG. 8a).

MAN-1 has also been shown to inhibit binding of activated monocytes to immobilized human endothelial cells under shear-flow conditions. Monocytes (stimulated and unstimulated) were passed over immobilized human microvascular endothelial cells (HMEC) under venous flow conditions. Adherent cells were counted. MAN-1 inhibits the binding of the activated Mac-1 population, whereas an unspecific CD11b antibody (clone 2LPM19c) inhibits activated and non-activated Mac-1 receptors (FIG. 14)

### Inhibition of Mac-1-Mediated Adhesion by Man-1-Derived Peptides Under Flow Conditions.

Since specificity of the scFv clones generated from the synthetic library is determined by the HCDR3 sequence, Applicant evaluated whether activation-specific blockade can be achieved by peptides derived from the HCDR3s of MAS-1 and MAS-2. To increase stability, synthesized cyclic peptides of the HCDR3 sequence were synthesized. CHO cells expressing either the native or the activated form of Mac-1 were perfused at an arterial flow rate over a fibrinogen-matrix. The MAS-1 as well as the MAS-2-derived peptides blocked adhesion of CHO cells, which express the activated form of Mac-1, to a significantly higher extent than the CHO cells, which express the native form of Mac-1 (FIG. 8c). This implies an activation-specific blockade of Mac-1 by the HCDR3-derived peptides.

### Example 7

### Man-1 as a Diagnostic Probe for Sepsis

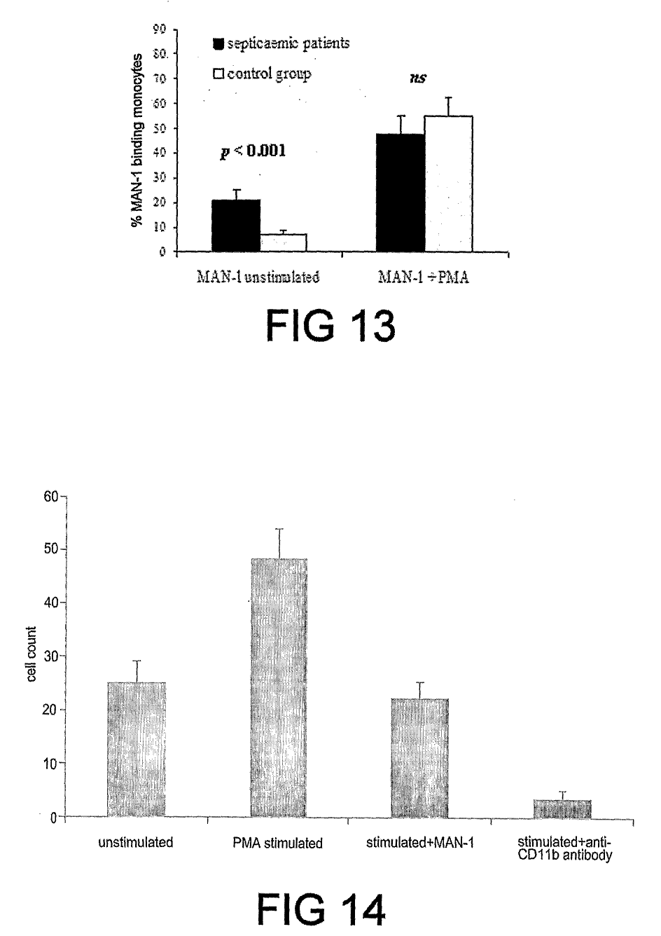

To demonstrate the feasibility of activation-specific anti-Mac-1 scFvs as diagnostic tools, 18 patients with severe sepsis were investigated. In comparison to an age- and sex-matched control group without clinical or laboratory signs of inflammation, patients with sepsis demonstrate a significantly increased binding of MAN-1 to peripheral blood monocytes (FIG. 13). Upon PMA stimulation no significant difference in MAN-1 binding to monocytes could be observed between the two groups (FIG. 13), implying that the stimulatory capacity of monocytes is preserved in both groups. However in the patients with sepsis monocytes are clearly preactivated. Thus, MAN-1 may be implemented as a diagnostic tool for the detection of monocyte activation in sepsis.

Finally, it is to be understood that various other modifications and/or alterations may be made without departing from the spirit of the present invention as outlined herein.

1. A non-natural molecule capable of binding to activated Mac-1.

2-29. (canceled)

30. A molecule according to claim 1 capable of binding to the I-domain of activated Mac-1.

31. A molecule according to claim 1 that is substantially incapable of binding to non-activated Mac-1.

32. A molecule according to claim 1 substantially incapable of binding to an integrin that is not Mac-1.

33. A molecule according to claim 1 capable of interfering with the binding of a natural ligand to Mac-1.

34. A molecule according to claim 33 wherein the natural ligand is selected from the group consisting of intracellular adhesion molecule-1 (ICAM-1), fibrinogen (Fg), Factor Xa, heparin, GPIb-alpha, JAM-3, lipoprotein (a), and a denatured protein.

35. A molecule according to claim 1 substantially incapable of interfering with the binding of C3bi to Mac-1.

36. A molecule according to claim 1, wherein the molecule is a peptide, polypeptide or derivative thereof including the amino acid sequence motif DX₁X₂X₃X₄X₅X₆ X₇X₈X₉Y, wherein X₁ is S or no amino acid; X₂ is independently T, L or F; X₃ is independently L or W; X₄ is independently A or G; X₅ is independently P, F or no amino acid; X₆ is Q or no amino acid; X₇ is independently I, L or S; X₈ is independently F or Y; and X₉ is independently E or D.

37. A peptide, polypeptide or derivative thereof according to claim 36 including the amino acid sequence motif DLWGFQLFDY, DFWGSYDY or DSTLAPIFEY or equivalent sequence.

38. A peptide, polypeptide or derivative thereof according to claim 36 in the form of a single-chain antibody molecule.

39. A peptide, polypeptide or derivative thereof according to claim 38 including one or more of the following regions: HCDR1, HCDR2, HCDR3, LINKER, LCDR1, LCDR2, LCDR3.

40. A peptide, polypeptide or derivative thereof according to claim 39 wherein the HCDR1 is AASGFIFRDYDMD or AASGFSNYGIH or equivalent sequence, the HCDR2 is independently RSTKRTSSYTIQDAA or VALISYDNGNKKFYA or equivalent sequence, the HCDR3 region is DLWGFQLFDY, DFWGSYDY or DSTLAPIFEY or equivalent sequence, the LINKER is independently KLEEGEFSEARV or equivalent sequence, the LCDR1 is independently GGNNIGSKSVH or GGNNIGSTTVH or equivalent sequence, the LCDR2 is independently YDSVRPS or DDNERPS or equivalent sequence, the LCDR3 is independently QVWDSNTDHYV or QVWDSGSDHVV or equivalent sequence.

41. A composition including a molecule, peptide or polypeptide or derivative thereof according to claim 1 and a pharmaceutically acceptable carrier.

42. A method of treating or preventing a Mac-1 mediated condition, the method including administering to a subject in need thereof an effective amount of a composition according to claim 41.

43. A method according to claim 42 wherein the condition is an inflammatory condition.

44. A method according to claim 42 wherein the condition is selected from the group consisting Crohn's disease, colitis ulcerosa, multiple sclerosis, sarcoidosis, psoriasis, atherosclerosis and its clinical sequelae, scleroderma, intestinal adhesions, hypertrophic scars, rheumatoid arthritis, septicemia, autoimmune disease, acute coronary syndrome, HIV infection, and ischemia and reperfusion injuries, neointimal thickening, infiltration of polymorpholeucocytes, autoimmune disease, and neovascularisation-mediated diseases.

45. A method for detecting the presence, absence or level of an activated Mac-1 in a subject or a test article, the method including exposing the subject, or a biological sample of the subject or the test article, to a molecule according to claim 1, and detecting binding of the molecule to activated Mac-1.

46. A method according to claim 45 wherein the step of detecting binding involves use of a labeled or tagged molecule according to claim 1.

47. A method of diagnosis or prognosis of a Mac-1 mediated condition, including a method according to claim 45.

48. A method according to claim 47 wherein the Mac-1 mediated condition is sepsis.

49. A method according to claim 46 wherein the tag or label is a radioisotope.

50. A method according to claim 46 wherein the tag or label is paramagnetic.

51. A method according to claim 46 wherein the tag or label is a fluorophore.

52. A method according to claim 46 wherein the presence, absence or level of the tagged molecule, peptide, polypeptide or derivative thereof is detected by a diagnostic imaging technique.

53. A method according to claim 52 wherein the diagnostic imaging technique is selected from the group consisting of MRI, flow cytometry, ultrasound, gamma scintigraphy, computer tomography and near-infrared detection.

54. A method for identifying a molecule capable of binding to activated Mac-1, the method including the steps of providing a library of candidate molecules, providing a first cell type exhibiting either activated Mac-1 or non-activated Mac-1, providing a second cell type exhibiting either activated Mac-1 or non-activated Mac-1, exposing the library of candidate molecules to the first cell type exhibiting non-activated Mac-1 and removing bound molecules to leave a first pool of molecules, exposing the first pool of molecules to the first cell type exhibiting activated Mac-1 and removing unbound molecules to leave a second pool of molecules, exposing the second pool of molecules to the second cell type exhibiting non-activated Mac-1 and removing unbound molecules to leave a third pool of molecules, exposing the third pool of molecules to the second cell type exhibiting activated Mac-1 and removing the unbound molecules to leave a fourth pool of molecules.

55. A molecule, peptide or polypeptide or derivative thereof identified by a method according to claim 54.

56. A molecule according to claim 1 substantially as hereinbefore described by reference to any of the noncomparative Figures or Examples.
